(19) United States
(12) Patent Application Publication
(10) Pub. No.: US 2026/0325664 A1
(43) Pub. Date: Oct. 22, 2026

(54) CPG Conjugates and Methods of Use

(71) Applicant: The Board of Trustees of the Leland Stanford Junior University, Stanford, CA (US)

(72) Inventors: Caitlyn Miller, Standford, CA (US); Jennifer R. Cochran, Stanford, CA (US); Carolyn R. Bertozzi, Menlo Park, CA (US)

(21) Appl. No.: 19/483,832

(22) PCT Filed: May 15, 2024
(86) PCT No.: PCT/US2024/029498

§ 371 Date: Nov. 12, 2025

Related U.S. Application Data
(60) Provisional application No. 63/466,896, filed on May 16, 2023.

(51) Int. Cl. A61K 47/68; A61K 47/64; A61K 47/65; A61P 37/04; C07K 16/28

(52) U.S. Cl. CPC A61K 47/6807; A61K 47/64; A61K 47/65; A61K 47/6849; A61K 47/6889; A61P 37/04; C07K 16/2818; C07K 16/2827; C07K 16/2842

(57) ABSTRACT

Provided are conjugates comprising a targeting moiety or antigen conjugated to a CpG oligonucleotide. In some instances, the targeting moiety or antigen conjugated to the 3′ end region of a CpG oligonucleotide. According to some embodiments, the CpG oligonucleotide comprises a palindromic sequence and/or is one which, in its unconjugated state, activates NF-κB and interferon regulatory factor (IRF) pathways. Compositions comprising the conjugates, as well as methods of using the conjugates, are also provided.

### CROSS-REFERENCE TO RELATED APPLICATIONS

This application claims priority to U.S. Provisional Application No. 63/466,896 filed May 16, 2023, which is incorporated by reference herein in its entirety.

### SEQUENCE LISTING

The present application contains a Sequence Listing, which has been submitted electronically in XML format and is hereby incorporated by reference in its entirety. Said XML copy, created on May 14, 2024, is named STAN-2067WO_SEQLIST, and is 44,264 bytes in size.

### INTRODUCTION

Specific modulation of the immune system is a common objective of many therapeutics across various disease types, including cancers, infectious diseases, allergies, and autoimmune disorders. CpG oligonucleotides are synthetic DNA oligonucleotides containing unmethylated “CpG” motifs that specifically activate the pathogen-recognition receptor “Toll-like receptor 9 (TLR9)” and thus, are useful immunomodulators for many applications ranging from vaccine adjuvants (for infectious diseases and cancer) to cancer immunotherapy and allergen-specific immunotherapy.

CpG binding to TLR9 can lead to activation of two downstream signaling pathways: NF-κB and interferon regulatory factor (IRF) pathways (FIG. 1). Activation of the NF-κB pathway leads to the induction of proinflammatory cytokines (e.g., IL-6, TNF-α) as well as prominent B cell activation and proliferation, whereas activation of the IRF pathway (particularly IRF7) primarily leads to the production of type I interferons (e.g., IFNα).

Different classes of CpG oligonucleotides have been developed that have different sequence motifs/properties (which affect their secondary structure and ability to form self-dimers/multimers) and different DNA backbone properties (phosphodiester or phosphodiester/phosphorothioate hybrid vs. fully phosphorothioate). There are three main classes of CpG: Class A primarily activates IRF signaling (particularly IRF7) and is a strong inducer of IFNα, Class B primarily stimulates NF-κB activation and results in proinflammatory cytokine induction, and Class C can activate both pathways, leading to production of both proinflammatory cytokines and IFNα.

For many applications, it can be desirable to attach CpG oligonucleotides to another molecule to form a conjugate. For example, attaching CpG to a tumor-targeting agent (e.g., targeting peptide, protein, or antibody) can be useful for improving delivery to tumors for cancer immunotherapy. Alternatively, for vaccines, it may be desirable to covalently attach CpG to an antigenic peptide or protein.

Across the published literature about CpG conjugates, Class B oligonucleotides, which are linear and monomeric, have historically been the most prolific given that they can be used to create much more homogeneous conjugate products than Class A CpG, which form higher order multimers. Class C CpG oligonucleotides are also desirable for developing conjugates, but this class of CpG oligonucleotides was discovered later than Class A and Class B, and there is much less available literature about Class C CpG conjugates and how the conjugation site may affect their behavior.

Class A CpG oligonucleotides stimulate the interferon regulatory factor (IRF) immune pathway, whereas class B oligonucleotides stimulate the NF-kB immune pathway. In contrast to Class A and Class B, Class C CpG oligonucleotides can effectively stimulate both immune pathways (NF-kB and IRF). Unexpectedly, we found that the conjugation site could, in fact, affect the two immune pathways in different ways for conjugates using class C CpG oligonucleotides, specifically. These surprising results helped us determine which conjugation site should be selected when developing Class-C CpG conjugates to ensure the conjugate can properly activate both immune pathways.

### SUMMARY

Provided are conjugates comprising a targeting moiety or antigen conjugated to a CpG oligonucleotide. In some instances, the targeting moiety or antigen is conjugated to the 3′ end region of a CpG oligonucleotide. According to some embodiments, the CpG oligonucleotide comprises palindromic sequences and/or is one which, in its unconjugated state, activates NF-κB and interferon regulatory factor (IRF) pathways. Compositions comprising the conjugates, as well as methods of using the conjugates, are also provided.

Various additional embodiments of the invention include the following.

Embodiment 1 is a conjugate comprising: a targeting moiety or antigen conjugated via a linker to the 3′ end region of a CpG oligonucleotide, wherein the CpG oligonucleotide is one which, in its unconjugated state, activates NF-κB and interferon regulatory factor (IRF) pathways.

Embodiment 2 is the conjugate of embodiment 1, wherein the CpG oligonucleotide comprises palindromic sequences.

Embodiment 3 is the conjugate of embodiment 2, wherein the palindromic sequence comprises a first sequence and a second sequence, wherein the second sequence is a reverse complement of the first sequence and wherein the first sequence and second sequence are each from 4 to 50, 4 to 25, 4 to 20, 5, 6, 7, 8, 9, 10, 11, 12, 13, or 14 nucleotides long.

Embodiment 4 is the conjugate of any one of embodiments 1 to 3, wherein the CpG oligonucleotide comprises from 10 to 100, from 10 to 50, or from 20 to 40 nucleotides.

Embodiment 5 is the conjugate of any one of embodiments 1 to 4, wherein the CpG oligonucleotide comprises CpG dinucleotides with 1, 2, 3, 4, 5, 6, or 7 intervening nucleotides between each CpG dinucleotide, independently.

Embodiment 6 is the conjugate of any one of embodiments 1 to 5, wherein the targeting moiety or antigen is conjugated to the 5th nucleotide, the 4th nucleotide, the 3rd nucleotide, or the 2nd nucleotide from the 3′ end, or the last nucleotide on the 3′ end of the CpG oligonucleotide.

Embodiment 7 is the conjugate of embodiment 6, wherein the targeting moiety or antigen is conjugated to the last nucleotide on the 3′ end of the CpG oligonucleotide.

Embodiment 8 is the conjugate of any one of embodiments 1 to 7, wherein the CpG oligonucleotide comprises one or more internucleotide linkages resistant to nuclease degradation.

Embodiment 9 is the conjugate of embodiment 8, wherein the one or more internucleotide linkages resistant to nuclease degradation comprise a phosphorothioate linkage, a boranophosphate linkage, a phosphorodiamidate linkage, a phosphoamidate linkage, a thiophosphoramidate linkage, or any combination thereof.

Embodiment 10 is the conjugate of embodiment 8, wherein the one or more internucleotide linkages resistant to nuclease degradation comprise one or more phosphorothioate linkages.

Embodiment 11 is the conjugate of any one of embodiments 1 to 10, wherein the CpG oligonucleotide is a class-C CpG oligonucleotide.

Embodiment 12 is the conjugate of any one of embodiments 1 to 11, wherein the CpG oligonucleotide comprises the following nucleotide sequence: 5′-TCGAACGTTCGAACGTTCGAACGTTCGAAT-3′. (SEQ ID NO: 1).

Embodiment 13 is the conjugate of any one of embodiments 1 to 11, wherein the CpG oligonucleotide comprises the following nucleotide sequence: 5′-TCGTCGTCGTTCGAACGACGTTGAT-3′. (SEQ ID NO: 2).

Embodiment 14 is the conjugate of any one of embodiments 1 to 13, comprising a targeting moiety conjugated to the 3′ end region of a CpG oligonucleotide.

Embodiment 15 is the conjugate of embodiment 14, wherein the targeting moiety is chosen from: a polypeptide, a ligand, an aptamer, a nanoparticle, and a small molecule.

Embodiment 16 is the conjugate of embodiment 15, wherein the targeting moiety is a polypeptide.

Embodiment 17 is the conjugate of embodiment 16, wherein the targeting moiety comprises an antibody.

Embodiment 18 is the conjugate of embodiment 17, wherein the antibody is an IgG, single chain Fv (scFv), Fab, (Fab)2, (scFv′)2, or a single variable domain located on a heavy chain (VHH).

Embodiment 19 is the conjugate of any one of embodiments 14 to 18, wherein the targeting moiety specifically binds a cell surface molecule.

Embodiment 20 is the conjugate of any one of embodiments 14 to 19, wherein the targeting moiety specifically binds to a tumor antigen on the surface of the cancer cell.

Embodiment 21 is the conjugate of embodiment 20, wherein the tumor antigen is αvβ1 integrin, αvβ3 integrin, αvβ5 integrin, αvβ6 integrin, α5β1 integrin, 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAPi), tissue factor (TF), T cell immunoglobulin and mucin protein-1 (TIM-1), Tn antigen, trophoblast cell-surface antigen (TROP-2), Wilms' tumor 1 (WT1), or VEGF-A, or any combination thereof.

Embodiment 22 is the conjugate of any one of embodiments 14 to 21, wherein the targeting moiety specifically binds αvβ1 integrin, αvβ3 integrin, αvβ5 integrin, αvβ6 integrin, α5β1 integrin, or any combination thereof.

Embodiment 23 is the conjugate of any one of embodiments 14 to 22, wherein the targeting moiety is a knottin peptide.

Embodiment 24 is the conjugate of embodiment 23, wherein the knottin peptide comprises an EETI-II peptide, an AgRP peptide, a ω-conotoxin peptide, a Kalata B1 peptide, an MCoTI-II peptide, an agatoxin peptide, or a chlorotoxin peptide.

Embodiment 25 is the conjugate of embodiment 23 wherein the knottin peptide comprises an EETI-II peptide.

Embodiment 26 is the conjugate of any one of embodiments 23 to 25, wherein the knottin peptide is an EETI-based integrin-binding peptide comprising GCX₁X₂X₃X₄X₅X₆X₇X₈X₉X₁₀X₁₁X₁₂X₁₃X₁₄X₁₅X₁₆X₁₇X₁₈X₁₉X₂₀CX₂₁DSDCX₂₂AGCVCGPNGX₂₃CG (SEQ ID NO: 19) wherein X₁-X₃ are any amino acid; X₄-X₂₀, if present, are any amino acid; and wherein X₂₁-X₂₃ are any amino acid. Any amino acid may include standard or unnatural amino acids.

Embodiment 27 is the conjugate of any one of embodiments 23 to 26, wherein the knottin peptide comprises an integrin binding loop comprising PRPRGDNPPLT (SEQ ID NO: 22) or PQGRGDWAPTS (SEQ ID NO: 23).

Embodiment 28 is the conjugate of any one of embodiments 14 to 27, wherein the targeting moiety comprises a ligand, a nanoparticle, or a small molecule.

Embodiment 29 is the conjugate of any one of embodiments 1 to 13, comprising an antigen conjugated to the 3′ end region of a CpG oligonucleotide.

Embodiment 30 is the conjugate of embodiment 29, wherein the antigen is a microbial antigen.

Embodiment 31 is the conjugate of embodiment 29, wherein the microbial antigen is a bacterial antigen.

Embodiment 32 is the conjugate of embodiment 29, wherein the antigen is a viral antigen.

Embodiment 33 is the conjugate of embodiment 29, wherein the antigen is a tumor antigen.

Embodiment 34 is the conjugate of embodiment 29, wherein the antigen is an allergen.

Embodiment 35 is the conjugate of any one of embodiments 1 to 34, wherein the linker is a non-cleavable linker.

Embodiment 36 is the conjugate of any one of embodiments 1 to 34, wherein the linker is a cleavable linker.

Embodiment 37 is the conjugate of embodiment 36 wherein the linker is an enzymatically cleavable linker.

Embodiment 38 is a composition comprising the conjugate of any one of embodiments 1 to 37.

Embodiment 39 is the composition of embodiment 38, wherein the composition is formulated for administration to a subject in need thereof.

Embodiment 40 is the composition of embodiment 39, comprising the conjugate of any one of embodiments 20 to 22.

Embodiment 41 is a method comprising administering to a subject in need thereof an effective amount of the composition of embodiment 39.

Embodiment 42 is a method of treating cancer in a subject in need thereof, the method comprising administering to the subject the composition of embodiment 40 in an amount effective to treat the cancer.

Embodiment 43 is a method of embodiment 42, wherein the cancer is chosen from a solid tumor, optionally a blastoma, carcinoma, lymphoma, or sarcoma,

Embodiment 44 is the method according to embodiment 41 to 43, wherein the subject is receiving an immune checkpoint inhibitor therapy.

Embodiment 45 is the method according to embodiment 44, wherein the immune checkpoint inhibitor therapy comprises administration to the subject of a therapeutically effective amount of an inhibitor of B7-H3, CTLA-4, LAG-3, PD-1, PD-L1, TIGIT, TIM-3, VISTA, or any combination thereof.

Embodiment 46 is the method according to embodiment 45, wherein the immune checkpoint inhibitor therapy comprises administration to the subject of a therapeutically effective amount of a PD-1 inhibitor.

Embodiment 47 is the method according to embodiment 46, wherein the PD-1 inhibitor is an anti-PD-1 antibody.

Embodiment 48 is the method according to embodiment 47, wherein the anti-PD-1 antibody is camrelizumab, cemiplimab, dostarlimab, nivolumab, pembrolizumab, pidilizumab, pimivalimab, retifanlimab, sintilimab, spartalizumab, tislelizumab, or toripalimab.

Embodiment 49 is the method according to any one of embodiments 44 to 48, wherein the immune checkpoint inhibitor therapy comprises administration to the subject of a therapeutically effective amount of a PD-L1 inhibitor.

Embodiment 50 is the method according to embodiment 49, wherein the PD-L1 inhibitor is an anti-PD-L1 antibody.

Embodiment 51 is the method according to embodiment 50, wherein the anti-PD-L1 antibody is atezolizumab, avelumab, or durvalumab.

Embodiment 52 is the method according to any one of embodiments 44 to 51, comprising administering to the subject the immune checkpoint inhibitor therapy.

Embodiment 53 is an immunogenic composition comprising the conjugate of any one of embodiments 29 to 34, and a pharmaceutically acceptable carrier.

Embodiment 54 is a method of inducing an immune response in a subject, the method comprising administering the immunogenic composition of embodiment 53 to the subject an amount effective to induce an immune response in the subject.

### BRIEF DESCRIPTION OF THE FIGURES

FIG. 1: Schematic illustration of TLR9 activation pathways.

FIG. 2: General structure of 5′ Amino-CpG and 3′ Amino-CpG oligonucleotides.

FIG. 3: Synthesis strategies for producing 5′ DBCO-CpG and 3′ DBCO-CpG.

FIG. 4: Synthesis strategy for producing 5′ PIP-CpG (PIP peptide sequence is SEQ ID NO:44).

FIG. 5: Synthesis strategy for producing 3′ PIP-CpG (PIP peptide sequence is SEQ ID NO:44).

FIG. 6: RAW-Blue assay comparing 24 hour treatment of CpG-C SD101 and 5′ Amino CpG (CpG-C SD101).

FIG. 7: IL-6 production from human PBMCS after 24 h incubation with CpG-C SD101 or 5′ PIP-CpG (CpG-C SD101) and schematics of these two treatments.

FIG. 8: CpG-C SD101 and 5′ PIP-CpG (CpG-C SD101) stimulate similar levels of proinflammatory cytokines (IL-6, TNF, IL-1β, IL-10) in human PBMCs after 24 h incubation.

FIG. 9: IFNα production from human PBMCs following 24 h stimulation with 3′ PIP-CpG (CpG-C SD101), CpG-C SD101, 5′ PIP-CpG (CpG-C SD101), 5′ DBCO-CpG (CpG-C SD101), and 5′ Amino-CpG (CpG-C SD101) and comparison of structure of each CpG modification.

FIG. 10: CpG-C SD101 and 3′ PIP-CpG (CpG-C SD101) stimulate similar levels of IL-6 in human PBMCs after 24 h incubation.

FIG. 11: RAW-Blue assay comparing 24-hour treatment with CpG-C SD101, 5′ PIP-CpG (CpG-C SD101), and 3′ PIP-CpG (CpG-C SD101).

FIG. 12: Comparison of IFNα and IL-6 production from human PMBCs after 24 h for example class A, B, and C CpG oligonucleotides.

FIG. 13: Comparison of IL-6 production from human PBMCs after 24 h incubation with 500 nM or 1000 nM of free class A, B, or C CpG oligonucleotides versus their corresponding 5′ PIP CpG conjugates.

FIG. 14: Comparison of IFNα production from human PMBCs after 24 h incubation with 500 nM and 1000 nM free class A, B, or C CpG oligonucleotides versus their corresponding 5′ PIP CpG conjugate.

FIG. 15: Comparison of IL-6 and IL-10 production in human PBMCs after 24 hours of treatment with CpG-C M362 and 5′ PIP-CpG (CpG-C M362).

FIG. 16: Comparison of IFNα production in human PBMCs after 24 hours of treatment with CpG-C M362 and 5′ PIP-CpG (CpG-C M362).

FIG. 17: Structures of 5′ PIP-CpG (CpG-C SD101), 3′ PIP-CpG (CpG-C SD101), and 3′ PIP-PS-CpG (CpG-C SID101).

FIG. 18: Comparison of human, mouse, and cynomolgus monkey (cyno) plasma stability over 72 hours at 37° C. for 3′ PIP-PS-CpG, 5′ PIP-CpG, and 3′ PIP-CpG, wherein the CpG used in this experiment was CpG-C SD101.

FIG. 19: Comparison of IFNα production in human PBMCs after 24 hours of treatment with CpG, 5′ PIP-CpG, 3′ PIP-PS-CpG, 3′ PIP-CpG, and PIP, wherein the CpG used in this experiment was CpG-C SD101.

FIG. 20: Comparison of IL-6 production in human PBMCs after 24 hours of treatment with CpG, 5′ PIP-CpG, and 3′ PIP-CpG, wherein the CpG used in this experiment was CpG-C SD101.

FIG. 21: Comparison of IL-6 production in human PBMCs after 24 hours of treatment with 3′ PIP-CpG, 3′ PIP-PS-CpG, and PIP, wherein the CpG used in this experiment was CpG-C SD101.

FIG. 22: Synthesis strategy for producing 3′ MMPa-CpG and 3′ MMPb-CpG.

FIG. 23: Synthesis strategy for producing 3′ DBCO-MMPa-CpG and 3′ DBCO-MMPb-CpG.

FIG. 24: Synthesis strategy for producing 3′ PIP-MMPa-CpG and 3′ PIP-MMPb-CpG.

FIG. 25: IFNα production from human PBMCs following 24 h stimulation with CpG, 3′ PIP-CpG, 3′ PIP-MMPa-CpG, 3′ PIP-MMPb-CpG, and 5′ PIP-CpG, wherein the CpG used in this experiment was CpG-C SD101.

FIG. 26: RAW-Blue assay comparing CpG, 3′ PIP-CpG, 3′ PIP-MMPa-CpG, and 3′ PIP-MMPb-CpG, wherein the CpG used in this experiment was CpG-C SD101.

FIG. 27: DNA PAGE gel showing that 3′ PIP-MMPa-CpG (enzyme-cleavable linker shown is SEQ ID NO:45) and 3′ PIP-MMPb-CpG (enzyme-cleavable linker shown is SEQ ID NO:46) conjugates are cleaved by human MMP2 (hMMP2), mouse MMP2 (mMMP2), human MMP9 (hMMP9), and mouse MMP9 (mMMP9), wherein the CpG used in this experiment was CpG-C SD101.

FIG. 28: Competition binding assay results for PIP, 3′ PIP-CpG, 3′ PIP-MMPa-CpG, 3′ PIP-MMPb-CpG, and 5′ PIP-CpG in human PANC1 pancreatic cancer cells, wherein the CpG used in this experiment was CpG-C SD101.

### DESCRIPTION OF THE SEQUENCES

Table 1 provides a listing of certain sequences referenced herein. For SEQ ID Nos: 1-16, (1) each uppercase letter represents a nucleotide linked to its 3′-adjacent nucleotide (if present) by a phosphorothioate linkage; (2) each lowercase letter represents a nucleotide linked to its 3′-adjacent nucleotide (if present) by a phosphodiester linkage; and (3) bold regions show palindromic sequence regions. Italic font shows unmethylated CpG motifs (CG dinucleotides). For SEQ ID Nos: 17-19; 25-37; and 42-43, (1) each bold letter represents a member of the integrin binding loop; (2) Z=5-azido-L-norvaline.

TABLE 1
Description of Sequences

| | | |
|---|---|---|
| | | SEQ ID |
| Description | Sequences | NO |

Certain Class C CpG DNA Oligonucleotide Sequences

| | | |
|---|---|---|
| CpG-C | 5′-TCGAACGTTCGAACGTTCGAACGTTCGAAT-3′ | 1 |
| SD101 | | |
| CpG-C | 5′-TCGTCGTCGTTCGAACGACGTTGAT-3′ | 2 |
| M362 | | |
| CpG-C | 5′-TCGTCGTTTTCGGCGCGCGCCG-3′ | 3 |
| 2395 | | |
| CpG-C | 5′-TCGTAACGTTCGAACGTTCGAAC-3′ | 4 |
| DV281 | | |
| CpG-C | 5′-TCGTCGAACGTTCGAGATGAT-3′ | 5 |
| C274 | | |
| CpG-C | 5′-TCGAACGTTCGAACGTTCGAACGTT-3′ | 6 |
| C695 | | |
| CpG-C D- | 5′-TCGCGAACGTTCGCCGCGTTCGAACGCGG-3′ | 7 |
| SL03 | | |

Certain Class B CpG DNA Oligonucleotide Sequences

| | | |
|---|---|---|
| CpG-B | 5′-TCGTCGTTTTGTCGTTTTGTCGTT-3′ | 8 |
| 2006 | | |
| CpG-B | 5′-TGACTGTGAACGTTCGAGATGA-3′ | 9 |
| 1018 | | |
| CpG-B | 5′-TCCATGACGTTCCTGACGTT-3′ | 10 |
| 1826 | | |
| CpG-B | 5′-TCCATGACGTTCCTGATGCT-3′ | 11 |
| 1668 | | |

Certain Class A CpG DNA Oligonucleotide Sequences

| | | |
|---|---|---|
| CpG-A | 5′-GGtgcatcgatgcagGGGGG-3′ | 12 |
| C264 | | |
| CpG-A | 5′-GGgggacgatcgtcGGGGGG-3′ | 13 |
| 2216 | | |
| CpG-A | 5′-GGGgacgacgtcgtgGGGGGG-3′ | 14 |
| 2336 | | |
| CpG-A | 5′-GGggtcaacgttgaGGGGGG-3′ | 15 |
| 1585 | | |
| CpG-A G10 | 5′-gggggggggggacgatcgtcgggggggggg-3′ | 16 |

Certain EETI-based Knottin Peptide Sequences

| | | |
|---|---|---|
| EETI-II | GCPRILMRCKQDSDCLAGCVCGPNGFCG | 17 |
| 2.5F S15Z | GCPRPRGDNPPLTCZQDSDCLAGCVCGPNGYCG | 18 |
| F31Y | | |
| EETI- | GCX₁X₂X₃X₄X₅X₆X₇X₈X₉X₁₀X₁₁X₁₂X₁₃X₁₄X₁₅X₁₆X₁₇X₁₈X₁₉X₂₀ | 19 |
| based | CX₂₁QDSDCX₂₂AGCVCGPNGX₂₃CG, wherein X₁-X₃ are any amino | |
| Knottin | acid; X₄-X₂₀, if present, are any amino acid; and wherein | |
| Peptide | X₂₁-X₂₃ are any amino acid. Any amino acid may include | |
| | standard or unnatural amino acids. | |

Certain Integrin Binding Loops for EETI-based Knottin Peptide Sequences

| | | |
|---|---|---|
| FN-RGD1 | TGRGDSPASSK | 20 |
| binding | | |
| loop | | |
| FN-RGD2 | VTGRGDSPASS | 21 |
| binding | | |
| loop | | |
| PIP (2.5F) | PRPRGDNPPLT | 22 |
| binding | | |
| loop | | |
| 2.5D | PQGRGDWAPTS | 23 |
| binding | | |
| loop | | |
| 1.5B | TIGRGDWAPSE | 24 |
| binding | | |
| loop | | |

Additional EETI-based Knottin Peptide Sequences

| | | |
|---|---|---|
| 2.5F S15Z | GCPRPRGDNPPLTCZQDSDCLAGCVCGPNGFCG | 25 |
| 2.5F | GCPRPRGDNPPLTCSQDSDCLAGCVCGPNGFCG | 26 |
| 2.5F F31Y | GCPRPRGDNPPLTCSQDSDCLAGCVCGPNGYCG | 27 |
| 2.5F S15K | GCPRPRGDNPPLTCKQDSDCLAGCVCGPNGFCG | 28 |
| 2.5F S15K | GCPRPRGDNPPLTCKQDSDCLAGCVCGPNGYCG | 29 |
| F31Y | | |
| 2.5D | GCPQGRGDWAPTSCSQDSDCRAGCVCGPNGFCG | 30 |
| 2.5D F31Y | GCPQGRGDWAPTSCSQDSDCRAGCVCGPNGYCG | 31 |
| 2.5D S15Z | GCPQGRGDWAPTSCZQDSDCRAGCVCGPNGFCG | 32 |
| 2.5D S15Z | GCPQGRGDWAPTSCZQDSDCRAGCVCGPNGYCG | 33 |
| F31Y | | |
| 2.5D R21L | GCPQGRGDWAPTSCSQDSDCLAGCVCGPNGFCG | 34 |
| 2.5D R21L | GCPQGRGDWAPTSCSQDSDCLAGCVCGPNGYCG | 35 |
| F31Y | | |
| 2.5D S15Z | GCPQGRGDWAPTSCZQDSDCLAGCVCGPNGFCG | 36 |
| R21L | | |
| 2.5D S15Z | GCPQGRGDWAPTSCZQDSDCLAGCVCGPNGYCG | 37 |
| R21L F31Y | | |
| 2.5D S15K | GCPQGRGDWAPTSCKQDSDCRAGCVCGPNGFCG | 42 |
| 2.5D S15K | GCPQGRGDWAPTSCKQDSDCRAGCVCGPNGYCG | 43 |
| F31Y | | |

Certain other Knottin Peptide Sequences

| | | |
|---|---|---|
| Truncated | CVRLHESCLGQQVPCCDPAATCYCRFFNAFCYCR | 38 |
| AgRP | | |
| Kalata B1 | CGETCVGGTCNTPGCTCSWPVCTRNGLPV | 39 |
| McoTI-II | SGSDGGVCPKILKKCRRDSDCPGACICRGNGYCG | 40 |
| Chlorotoxin | MCMPCFTTDHQMARKCDDCCGGKGRGKCYGPQCLCR | 41 |

### DETAILED DESCRIPTION

Before the conjugates, compositions and methods of the present disclosure are described in greater detail, it is to be understood that the conjugates, compositions and methods are not limited to particular embodiments described, as such may, of course, vary. It is also to be understood that the terminology used herein is for the purpose of describing particular embodiments only, and is not intended to be limiting, since the scope of the conjugates, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the conjugates, compositions and methods. The upper and lower limits of these smaller ranges may independently be included in the smaller ranges and are also encompassed within the conjugates, compositions and methods, subject to any specifically excluded limit in the stated range. Where the stated range includes one or both of the limits, ranges excluding either or both of those included limits are also included in the conjugates, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

Unless defined otherwise, all technical and scientific terms used herein have the same meaning as commonly understood by one of ordinary skill in the art to which the conjugates, compositions and methods belong. Although any conjugates, compositions and methods similar or equivalent to those described herein can also be used in the practice or testing of the conjugates, compositions and methods, representative illustrative conjugates, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present conjugates, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the conjugates, compositions and methods, which are, for clarity, described in the context of separate embodiments, may also be provided in combination in a single embodiment. Conversely, various features of the conjugates, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present conjugates, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### 1. Conjugates

Aspects of the present disclosure include conjugates. In certain embodiments, a conjugate of the present disclosure comprises a targeting moiety or antigen conjugated via a linker to the 3′ end region of a CpG oligonucleotide.

### A. CpG Oligonucleotides

As used herein, a “CpG oligonucleotide” is a multimer of nucleotides (e.g., from 10 to 100 nucleotides in length, such as from 10 to 50 nucleotides in length) comprising unmethylated CpG dinucleotides (unmethylated cytosine-guanine dinucleotides). CpG oligonucleotides may be synthetic or may be made enzymatically, and, in some embodiments, are 10 to 50 (e.g., 20 to 40) nucleotides in length, for example. CpG oligonucleotides may comprise one or more CpG dinucleotide pairs. In some embodiments, the CpG oligonucleotides may be 25 to 35, 28 to 32, 29, 30, or 31 nucleotides in length, for example.

In some instances, the CpG oligonucleotide may be a palindromic sequence, i.e., the CpG oligonucleotide comprises a first sequence and a second sequence, wherein the second sequence is the complement in reverse order of the first sequence. The CpG oligonucleotide comprises CpG dinucleotides with or without intervening nucleotides between the CpG dinucleotide pairs. The first sequence and second sequence in the palindromic sequence may each be from 4 to 50, 4 to 25, 4 to 20, 5, 6, 7, 8, 9, 10, 11, 12, 13, or 14 nucleotides long. In some embodiments, there may be no intervening nucleotides, 1, 2, 3, 4, 5, 6, or 7 or more intervening nucleotides between each CG dinucleotide pair, independently. In some embodiments, the palindromic sequences align with the ends of the oligonucleotide and in other embodiments, one or both ends of the oligonucleotide are not part of the palindromic portion of the oligonucleotides (i.e., one or both ends comprise nonpalindromic portions).

According to some embodiments, the CpG oligonucleotide is one which, in its unconjugated state, activates NF-κB and interferon regulatory factor (IRF) pathways. The conjugates of the present disclosure are based in part on the unexpected finding that the site of conjugation on CpG oligonucleotides can influence the downstream immunological signaling pathways in useful ways. This is demonstrated herein using a class-C CpG, which are known to activate both NF-κB and IRF signaling. For example, demonstrated herein is that peptide conjugation to the 5′ or 3′ end regions of a Class C CpG oligonucleotide retains full TLR9-mediated NF-κB activation/pro-inflammatory cytokine production; however, only the peptide-CpG conjugate wherein the peptide was attached at the 3′ end region of the CpG oligonucleotide also retained full capacity for IFNα production, which is associated with TLR9-mediated IRF activation. Thus, while 5′ Class C CpG conjugates are still active and potent TLR9 agonists with full NF-κB activation capacity, their ability to induce IFNα production is selectively impaired. Furthermore, these findings were not specific to 5′ peptide-CpG conjugates given that even smaller chemical moieties attached to the 5′ end of the class C CpG also led to diminished IFNα production. In summary, these surprising findings establish that the 3′ end region is an optimal conjugation site on CpGs which, in their unconjugated states, activate both the NF-κB and IRF pathways (e.g., class-C CpGs and variants thereof)—when both NF-κB activation and IFNα production is desired.

As used herein, conjugation to the “3′ end region” of the CpG oligonucleotide means that the targeting moiety or antigen is conjugated via the linker to a nucleotide within 5 nucleotides, 4 nucleotides, 3 nucleotides, or 2 nucleotides of the 3′ terminal nucleotide of the CpG oligonucleotide, or via the linker to the 3′ terminal nucleotide of the CpG oligonucleotide. The last 5 nucleotides on the 3′ end of the CpG oligonucleotide comprise the 3′ end region. Thus, the conjugate may be conjugated to the 5^{th} nucleotide, the 4^{th} nucleotide, the 3^{rd} nucleotide, or the 2^{nd} nucleotide from the 3′ end, or the last nucleotide on the 3′ end of the CpG oligonucleotide. The terms “3′ end” and “3′ end region” are interchangeable unless specifically referencing the terminal nucleotide.

In certain embodiments, the CpG oligonucleotide comprises one or more internucleotide linkages resistant to nuclease degradation. For example, the CpG oligonucleotide may comprise a phosphorothioate linkage, a boranophosphate linkage, a phosphorodiamidate linkage, a phosphoamidate linkage, a thiophosphoramidate linkage, or any combination thereof. In some instances, the one or more internucleotide linkages resistant to nuclease degradation comprise one or more phosphorothioate linkages. According to some embodiments, the backbone of the CpG oligonucleotide comprises a mixture of phosphodiester and phosphorothioate linkages. In other embodiments, the entire backbone of the CpG oligonucleotide comprises phosphorothioate linkages. For example, in some instances, the CpG oligonucleotide is a class-C CpG oligonucleotide, such as SEQ ID Nos: 1-7, as listed in Table 1.

### B. Targeting Moieties

According to some embodiments, the conjugates of the present disclosure comprise a targeting moiety conjugated via the linker to the 3′ end region of a CpG oligonucleotide. Non-limiting examples of targeting moieties include a polypeptide (e.g., an antibody, a knottin peptide, or the like), a ligand, an aptamer, a nanoparticle, and a small molecule.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; and the like.

In certain embodiments, the targeting moiety specifically binds a target molecule, e.g., a cell surface molecule of a target cell (e.g., a cancer cell), or an extracellular or secreted target molecule. As used herein, a first molecule “specifically binds” or “preferentially binds” to a target if it binds with greater affinity, avidity, more readily, and/or with greater duration than it binds to other substances, e.g., in a sample. In certain embodiments, the targeting moiety “specifically binds” the target molecule if it binds to or associates with the target molecule with an affinity or Ka (that is, an association rate constant of a particular binding interaction with units of 1/M) of, for example, greater than or equal to about 10⁴ M 1. Alternatively, affinity may be defined as an equilibrium dissociation constant (KD) of a particular binding interaction with units of M (e.g., 10⁻² M to 10⁻¹³ M, or less). In certain aspects, specific binding means the targeting moiety binds to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the targeting moiety for the target molecule can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

### 1. Antibody Targeting Moieties

According to some embodiments, the targeting moiety is an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the target molecule (e.g., a cell surface molecule of a target cell), including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, single chain Fv (scFv), Fab, (Fab)₂, (scFv′)₂, or a single variable domain located on a heavy chain (VHH). According to some embodiments, the antibody is a VHH (sometimes referred to herein and elsewhere as a “nanobody”). The antibody may be detectably labeled, e.g., with an in vivo imaging agent, a radioisotope, an enzyme which generates a detectable product, a fluorescent protein, and the like.

An antibody included as the targeting moiety will vary based on the cell to be targeted. In some embodiments, the antibody specifically binds to an antigen on the surface of a target cell. Target cells of interest include, but are not limited to, cells that are relevant to a particular disease or condition, e.g., cancer or other disease or condition of interest. According to some embodiments, the target cell is selected from a cancer cell, an immune cell, and an endothelial cell. As such, in some embodiments, the target cells are cancer cells. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In certain embodiments, the cancer cell is a carcinoma cell.

According to some embodiments, when the target cell is a cancer cell, the targeting moiety specifically binds to a tumor antigen on the surface of the cancer cell. Non-limiting examples of tumor antigens to which the targeting moiety may specifically bind include αvβ1 integrin, αvβ3 integrin, αvβ5 integrin, αvβ6 integrin, α5β1 integrin, 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAPi), tissue factor (TF), T cell immunoglobulin and mucin protein-1 (TIM-1), Tn antigen, trophoblast cell-surface antigen (TROP-2), Wilms' tumor 1 (WT1), or VEGF-A.

Non-limiting examples of antibodies that specifically bind to tumor antigens which may be employed as a targeting moiety include abagovomab, abituzumab, adecatumumab, alemtuzumab, altumomab, amatuximab, anatumomab, arcitumomab, ascrinvacumab, bavituximab, bectumomab, bevacizumab, bivatuzumab, blinatumomab, blontuvetmab, brentuximab, brontictuzumab, cantuzumab, cantuzumab, capromab, catumaxomab, cetuximab, citatuzumab, cixutumumab, clivatuzumab, conatumumab, dacetuzumab, dalotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elotuzumab, emactuzumab, emibetuzumab, enfortumab, enoblituzumab, enoticumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, icrucumab, igovomab, imgatuzumab, indatuximab, inotuzumab, intetumumab, ipilimumab, iratumumab, isatuximab, labetuzumab, lexatumumab, lifastuzumab, lintuzumab, lorvotuzumab, lucatumumab, lumretuzumab, mapatumumab, margetuximab, matuzumab, milatuzumab, minretumomab, mitumomab, moxetumomab, nacolomab, naptumomab, narnatumab, necitumumab, nesvacumab, nimotuzumab, nofetumomab, obinutuzumab, ocaratuzumab, ofatumumab, olaratumab, onartuzumab, ontuxizumab, oportuzumab, oregovomab, otlertuzumab, panitumumab, parsatuzumab, patritumab, pemtumomab, pertuzumab, pinatuzumab, pintumomab, polatuzumab, pritumumab, racotumomab, radretumab, ramucirumab, rilotumumab, rituximab, robatumumab, satumomab, seribantumab, sibrotuzumab, siltuximab, simtuzumab, sofituzumab, solitomab, tacatuzumab, tamtuvetmab, taplitumomab, tarextumab, tenatumomab, teprotumumab, tigatuzumab, tositumomab, tovetumab, trastuzumab, tremelimumab, tucotuzumab, ublituximab, vandortuzumab, vantictumab, vanucizumab, veltuzumab, vesencumab, vorsetuzumab, votumumab, zalutumumab, or a tumor antigen-binding variant thereof. As used herein, “variant” is meant the antibody specifically binds to the particular antigen (e.g., HER2 for trastuzumab) but has fewer or more amino acids than the parental antibody (e.g., is a fragment (e.g., scFv) of the parental antibody), has one or more amino acid substitutions relative to the parental antibody, or a combination thereof.

In certain embodiments, the targeting moiety is an antibody approved by the United States Food and Drug Administration and/or the European Medicines Agency (EMA) for use as a therapeutic antibody (e.g., for targeting certain disease-associated cells in a patient, etc.), or a fragment thereof (e.g., a single-chain version of such an antibody, such as an scFv version of the antibody) that retains the ability to specifically bind the target antigen.

When an antibody is employed as the targeting moiety, the CpG oligonucleotide may be conjugated via the linker to any convenient portion of the antibody. In certain embodiments, the CpG oligonucleotide is conjugated via the linker to a light chain of the antibody, e.g., a kappa (κ) light chain or fragment thereof or a lambda (λ) light chain or fragment thereof. According to some embodiments, the antibody light chain or fragment thereof includes a light chain variable region (V_{L}). Such an antibody light chain or fragment thereof may further include an antibody light chain constant region (C_{L}) or fragment thereof. In certain embodiments, the antibody light chain or fragment thereof is a full-length antibody light chain—that is, an antibody light chain that includes a V_{L} and a C_{L}. In certain embodiments, the CpG oligonucleotide is conjugated via the linker to a V_{L} (if present) or a C_{L} (if present), e.g., at or near the N-terminus of a V_{L} or at or near the C-terminus of a C_{L}.

When an antibody is employed as the targeting moiety, the CpG oligonucleotide may be conjugated via the linker to a heavy chain or fragment thereof of the antibody. In certain embodiments, the antibody heavy chain or fragment thereof includes a y, a, b, E, or p antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain variable region (V_{H}). Such an antibody heavy chain or fragment thereof may further include a heavy chain constant region or fragment thereof. For example, when the antibody includes a heavy chain constant region or fragment thereof, the antibody heavy chain constant region or fragment thereof may include one or more of a C_{H}1 domain, C_{H}2 domain, and/or C_{H}3 domain. According to some embodiments, the antibody heavy chain is a full-length antibody heavy chain—that is, an antibody heavy chain that includes a V_{H}, a C_{H}1 domain, a C_{H}2 domain, and a C_{H}3 domain. In certain embodiments, the CpG oligonucleotide is conjugated via the linker to an Fc region of the antibody. According to some embodiments, the CpG oligonucleotide is conjugated via the linker to the antibody at or near the N-terminus of a V_{H} or at or near the C-terminus of a C_{H}3 domain.

### 2. Knottin Peptide Targeting Moieties

According to some embodiments, the targeting moiety is a knottin peptide. In some instances, the knottin peptide comprises an engineered loop that binds to a cell surface molecule. The type of knottin peptide employed in the conjugates of the present disclosure may vary. Non-limiting examples of a knottin peptide that may be employed include an EETI-II peptide, an AgRP peptide, a w-conotoxin peptide, a Kalata B1 peptide, an McoTI-II peptide, an agatoxin peptide, and a chlorotoxin peptide. In some embodiments, the knottin peptide comprises those provided in WO 2008/045252 and WO 2014/063012. The three-dimensional structure of a knottin peptide is minimally defined by a particular arrangement of three disulfide bonds. This characteristic topology forms a molecular knot in which one disulfide bond passes through a macrocycle formed by the other two intra-chain disulfide bridges. Although their secondary structure content is generally low, knottins share a small triple-stranded antiparallel β-sheet, which is stabilized by the disulfide bond framework. Folding and functional activity of knottins are often mediated by loop regions that are diverse in both length and amino acid composition. While three disulfide bonds are the minimum number that defines the fold of this family of peptides, knottins can also contain additional cysteine residues, yielding molecules with four or more disulfide bonds and additional constrained loops in their structure. The term “cystine” refers to a Cys residue in which the sulfur group is linked to another amino acid though a disulfide linkage; the term “cysteine” refers to the —SH (“half cystine”) form of the residue. Binding loop portions may be adjacent to cystines, such that there are no other intervening cystines in the primary sequence in the binding loop.

The knottin peptide may be a peptide described in the online KNOTTIN database (dsimb.inserm.fr/KNOTTIN/), which includes detailed amino acid sequence, structure, classification and function information for thousands of polypeptides identified as contain cystine-knot motifs. Knottins are found in a variety of plants, animals, insects and fungi.

The knottin peptide may be full-length (that is, the length of the wild-type peptide/polypeptide), the knottin peptide may be truncated relative to the length of the wild-type peptide/polypeptide, or the knottin peptide may include additional amino acids such that the peptide is greater in length relative to the length of the wild-type peptide/polypeptide.

According to certain embodiments, a knottin peptide is based on any one of an Ecballium elaterium trypsin inhibitor II (EETI-II) peptide, an agouti-related protein (AgRP) peptide, a ω-conotoxin peptide, a Kalata B1 peptide, an McoTI-II peptide, an agatoxin peptide, or a chlorotoxin peptide. In some embodiments, the knottin peptide is based on an Ecballium elaterium trypsin inhibitor II (EETI-II) peptide.

By “EETI” is meant Protein Data Bank Entry (PDB) 2ETI. Its entry in the KNOTTIN database is EETI-II. In some embodiments, a knottin peptide of a conjugate of the present disclosure is based on an EETI-II peptide having the following amino acid sequence:

(SEQ ID NO: 17)
GCPRILMRCKQDSDCLAGCVCGPNGFCG

An EETI-based knottin peptide, 2.5F S15Z F31Y, (sometimes referred to herein as “PIP”) having an engineered binding loop that binds to each of αvβ1 integrin, αvβ3 integrin, αvβ5 integrin, αvβ6 integrin, and α5β1 integrin, which may be employed in a conjugate of the present disclosure, has the following amino acid sequence (with the integrin-binding loop in bold), where Z=5-azido-L-norvaline:

(SEQ ID NO: 18)
GCPRPRGDNPPLTCZQDSDCLAGCVCGPNGYCG

In certain embodiments, a knottin peptide of a conjugate of the present disclosure is an EETI-based integrin-binding peptide having the following amino acid sequence structure:

(SEQ ID NO: 19)
GCX₁X₂X₃X₄X₅X₆X₇X₈X₉X₁₀X₁₁X₁₂X₁₃X₁₄X₁₅X₁₆X₁₇X₁₈X₁₉X₂₀
CX₂₁QDSDCX₂₂AGCVCGPNGX₂₃CG

wherein
X₁-X₃=any amino acid;
X₄-X₂₀=if present, any amino acid;
X₂₁=any amino acid;
X₂₂=any amino acid; and
X₂₃=any amino acid, wherein for each numbered X position, any amino acid may include standard or unnatural amino acids.

TABLE 2
Description of certain integrin-binding loops for
EETI-based Knottin Sequences

| | Binding Loop | |
|---|---|---|
| | Sequences (N to | |
| Description | C terminus) | SEQ ID NO |
| FN-RGD1 binding loop | TGRGDSPASSK | 20 |
| FN-RGD2 binding loop | VTGRGDSPASS | 21 |
| PIP (2.5F) binding loop | PRPRGDNPPLT | 22 |
| 2.5D binding loop | PQGRGDWAPTS | 23 |
| 1.5B binding loop | TIGRGDWAPSE | 24 |

In certain embodiments, a knottin peptide of a conjugate of the present disclosure is an EETI-based integrin-binding peptide having an amino acid sequence selected from the following sequences shown in Table 3 (with the integrin-binding loops in bold):

TABLE 3
Description of certain EETI-based Knottin Sequences

| Description | Sequences (N to C terminus) | SEQ ID NO |
|---|---|---|
| 2.5F S15Z F31Y | GCPRPRGDNPPLTCZQDSDCLAGCVCGPNGYCG | 18 |
| 2.5F S15Z | GCPRPRGDNPPLTCZQDSDCLAGCVCGPNGFCG | 25 |
| 2.5F | GCPRPRGDNPPLTCSQDSDCLAGCVCGPNGFCG | 26 |
| 2.5F F31Y | GCPRPRGDNPPLTCSQDSDCLAGCVCGPNGYCG | 27 |
| 2.5F S15K | GCPRPRGDNPPLTCKQDSDCLAGCVCGPNGFCG | 28 |
| 2.5F S15K F31Y | GCPRPRGDNPPLTCKQDSDCLAGCVCGPNGYCG | 29 |
| 2.5D | GCPQGRGDWAPTSCSQDSDCRAGCVCGPNGFCG | 30 |
| 2.5D F31Y | GCPQGRGDWAPTSCSQDSDCRAGCVCGPNGYCG | 31 |
| 2.5D S15Z | GCPQGRGDWAPTSCZQDSDCRAGCVCGPNGFCG | 32 |
| 2.5D S15Z F31Y | GCPQGRGDWAPTSCZQDSDCRAGCVCGPNGYCG | 33 |
| 2.5D R21L | GCPQGRGDWAPTSCSQDSDCLAGCVCGPNGFCG | 34 |
| 2.5D R21L F31Y | GCPQGRGDWAPTSCSQDSDCLAGCVCGPNGYCG | 35 |
| 2.5D S15Z R21L | GCPQGRGDWAPTSCZQDSDCLAGCVCGPNGFCG | 36 |
| 2.5D S15Z R21L F31Y | GCPQGRGDWAPTSCZQDSDCLAGCVCGPNGYCG | 37 |
| 2.5D S15K | GCPQGRGDWAPTSCKQDSDCRAGCVCGPNGFCG | 42 |
| 2.5D S15K F31Y | GCPQGRGDWAPTSCKQDSDCRAGCVCGPNGYCG | 43 |

In some embodiments, the knottin peptide is based on an agouti-related protein (AgRP) peptide. By “AGRP” is meant PDB entry 1HYK and KNOTTIN database entry SwissProt AGRP_HUMAN. AGRP is a 132 amino acid neuropeptide that binds to melanocortin receptors in the human brain and is involved in regulating metabolism and appetite. The biological activity of AgRP is mediated by its C-terminal cysteine knot domain, which contains five disulfide bonds, but a fully active 34 amino acid truncated AgRP that contains only four disulfide bonds has been developed. In certain aspects, a knottin peptide of a conjugate of the present disclosure is based on a truncated AGRP peptide having the following amino acid sequence:

(SEQ ID NO: 38)
CVRLHESCLGQQVPCCDPAATCYCRFFNAFCYCR

According to some embodiments, a knottin peptide of a conjugate of the present disclosure is based on a Kalata B1 peptide having the following amino acid sequence:

(SEQ ID NO: 39)
CGETCVGGTCNTPGCTCSWPVCTRNGLPV

In certain embodiments, a knottin peptide of a conjugate of the present disclosure is based on a McoTI-II peptide having the following amino acid sequence:

(SEQ ID NO: 40)
SGSDGGVCPKILKKCRRDSDCPGACICRGNGYCG

According to some embodiments, a knottin peptide of a conjugate of the present disclosure is based on a chlorotoxin peptide having the following amino acid sequence:

(SEQ ID NO: 41)
MCMPCFTTDHQMARKCDDCCGGKGRGKCYGPQCLCR

Sequences and structural (e.g., loop) information for EETI-II, AgRP, w-conotoxin, Kalata B1, McoTI-II, agatoxin, chlorotoxin, and other knottin peptides upon which the knottin peptides of the conjugates of the present disclosure may be based may be found in the PDB, the KNOTTIN database, and other protein databases.

The knottin peptide may include an engineered loop that binds to a cell surface molecule—that is, the loop is engineered to bind to a target molecule on the surface of a cell. Knottins contain three disulfide bonds interwoven into a molecular ‘knot’ that constrain loop regions to a core of anti-parallel β-sheets. Wild-type EETI, for example, is composed of 28 amino acids with three disulfide-constrained loops: loop 1 (the trypsin binding loop, residues 3-8), loop 2 (residues 10-14), and loop 3 (residues 22-26). Knottin family members, which include protease inhibitors, toxins, and antimicrobials, share little sequence homology apart from their core cysteine residues. As a result, their disulfide-constrained loops tolerate much sequence diversity, making knottins amenable for protein engineering applications where mutations need to be introduced into a protein without abolishing its three-dimensional fold.

The engineered loop may include amino acid substitutions, insertions, and/or deletions in an existing loop of the knottin peptide, or the engineered loop may be a loop added to the knottin protein. That is, the knottin peptide of the conjugate may include a loop in addition to the one or more loops present in the wild-type peptide. By combining directed evolution with computational covariance analysis, guidelines for introducing modifications (both in amino acid sequence and loop length) into the loop regions of the knottin scaffold have been elucidated. See, e.g., Lahti et al., “Interrogating and Predicting Tolerated Sequence Diversity in Protein Folds: Application to E. elaterium Trypsin Inhibitor-II Cystine-Knot Miniprotein”, (2009) PloS Comput. Biol. 5(9): e1000499.

### 3. Ligand Targeting Moieties

According to some embodiments, the targeting moiety is a ligand. As used herein, a “ligand” is a substance that forms a complex with a biomolecule in nature to serve a biological purpose. The ligand may be a substance selected from a circulating factor, a secreted factor, a cytokine, a growth factor, a hormone, a peptide, a polypeptide, a small molecule, and a nucleic acid, that forms a complex with the target molecule, e.g., a cell surface molecule on the surface of a target cell. In certain embodiments, when the targeting moiety is a ligand, the ligand is modified in such a way that complex formation with the target molecule occurs, but the normal biological result of such complex formation does not occur. In certain embodiments, the ligand is the ligand of a cell surface receptor present on a target cell.

### 4. Aptamer Targeting Moieties

In certain embodiments, the targeting moiety is an aptamer. By “aptamer” is meant a nucleic acid (e.g., an oligonucleotide) that has a specific binding affinity for the target molecule. Aptamers exhibit certain desirable properties for targeted delivery of the CpG oligonucleotide, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C). Aptamers that find use in the context of the present disclosure include those described in Zhu et al., “Nucleic acid aptamer-mediated drug delivery for targeted cancer therapy”, (2015) ChemMedChem 10(1):39-45; Sun et al., “Oligonucleotide aptamers: new tools for targeted cancer therapy”, (2014) Mol. Ther. Nucleic Acids 3:e182; and Zhang et al., “Tumor-Targeted Drug Delivery with Aptamers”, (2011) Curr. Med. Chem. 18(27):4185-4194.

### 5. Nanoparticle Targeting Moieties

According to some embodiments, the targeting moiety is a nanoparticle. As used herein, a “nanoparticle” is a particle having at least one dimension in the range of from 1 nm to 1000 nm, from 20 nm to 750 nm, from 50 nm to 500 nm, including 100 nm to 300 nm, e.g., 120-200 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain aspects, the nanoparticle includes on its surface one or more of the other targeting moieties described herein, e.g., antibodies, ligands, aptamers, small molecules, etc. Nanoparticles that find use in the context of the present disclosure include those described in Wang et al., “Targeting nanoparticles to cancer”, (2010) Pharmacol. Res. 62(2):90-99; Rao et al., “Chitosan-Decorated Doxorubicin-Encapsulated Nanoparticle Targets and Eliminates Tumor Reinitiating Cancer Stem-like Cells”, (2015) ACS Nano 9(6):5725-5740; and Byrne et al., “Active targeting schemes for nanoparticle systems in cancer therapeutics”, (2008) Adv. Drug Deliv. Rev. 60(15):1615-1626.

### 6. Small Molecule Targeting Moieties

In some embodiments, the targeting moiety is a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In certain aspects, the target molecule is a cell surface receptor for which the ligand is a small molecule, and the targeting moiety is the small molecule ligand (or a derivative thereof) of the receptor. Small molecules that find use as targeting moieties are known. As just one example, folic acid (FA) derivatives have been shown to effectively target certain types of cancer cells by binding to the folate receptor, which is overexpressed, e.g., in many epithelial tumors. See, e.g., Vergote et al., “Vintafolide: a novel targeted therapy for the treatment of folate receptor expressing tumors”, (2015) Ther. Adv. Med. Oncol. 7(4):206-218. In another example, the small molecule sigma-2 has proven to be effective in targeting cancer cells. See, e.g., Hashim et al., “Targeted pancreatic cancer therapy with the small molecule drug conjugate SW IV-134”, (2014) Molecular Oncology 8(5):956-967. Sigma-2 is the small molecule ligand for sigma-2 receptors, which are overexpressed in many proliferating tumor cells including pancreatic cancer cells. In certain embodiments, a small molecule is employed as the targeting moiety, and it has been demonstrated in the context of a small molecule drug conjugate (SMDC) that the small molecule is effective at targeting a drug to a target cell of interest by binding to a cell surface molecule on the target cell.

### C. Antigen Conjugates

In certain embodiments, e.g., when it is desirable to employ the conjugate as a vaccine, a conjugate of the present disclosure comprises an antigen conjugated via the linker to the 3′ end region of a CpG oligonucleotide. In some instances, the antigen is a microbial antigen. For example, the antigen may be a bacterial antigen. According to some embodiments, the antigen is a viral antigen. In certain embodiments, the antigen is a tumor antigen. In some instances, the antigen is an allergen.

### D. Conjugation Strategies

The term “conjugation” or “conjugated” generally refers to a chemical linkage, either covalent or non-covalent, usually covalent, that proximally associates one molecule of interest with a second molecule of interest. In certain embodiments, the targeting moiety or antigen is conjugated to the 3′ end region of the CpG oligonucleotide via a linker. If present, the linker molecule(s) may be of sufficient length to permit the CpG oligonucleotide and targeting moiety or antigen to allow some flexible movement between the CpG oligonucleotide and targeting moiety or antigen. In some embodiments, a conjugate of the present disclosure comprises a linker molecule which is 6 or greater atoms long, e.g., from 6 to 200 atoms, 6 to 175 atoms, 6 to 150 atoms, 6 to 125 atoms, 6 to 100 atoms, or 6 to 75 atoms, e.g., about 6 to 50 atoms long. Linker molecules may also be, e.g., aryl acetylene, ethylene glycol oligomers (e.g., containing 2-10 monomer units), diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest and may serve as a neutral tether between components. The ordinarily skilled artisan will recognize that design of conjugates can include linkers that are all or partially flexible, such that the linker can include a flexible linker as well as one or more portions that confer a less flexible structure.

According to some embodiments, the targeting moiety or antigen is conjugated to the 3′ end region of the CpG oligonucleotide via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the targeting moiety or antigen is conjugated to the 3′ end region of the CpG oligonucleotide via a cleavable linker. According to some embodiments, the cleavable linker is a chemically labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically labile linkers include, but are not limited to, hydrazone-based linkers, oxime-based linkers, carbonate-based linkers, ester-based linkers, etc. In certain embodiments, the linker is an enzyme-cleavable linker, such as an enzyme-cleavable linker that is stable in the bloodstream but undergoes enzymatic cleavage upon internalization into a target cell, e.g., by a lysosomal protease (such as cathepsin or plasmin) in a lysosome of the target cell, e.g., a cancer cell. Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. According to some embodiments, the linker is an enzyme-cleavable linker, such as an enzyme-cleavable linker that is stable in the bloodstream but undergoes enzymatic cleavage extracellularly, e.g., by a matrix metalloprotease (MMP—non-limiting examples of which include MMP2 and MMP9), e.g., in a tumor microenvironment.

Chemically labile linkers, enzyme-labile, and non-cleavable linkers are known and described in detail, e.g., in Ducry & Stump, “Antibody-drug conjugates: linking cytotoxic payloads to monoclonal antibodies”, (2010) Bioconjugate Chem. 21:5-13; Nolting, B., “Linker technologies for antibody-drug conjugates”, (2013) Methods Mol Biol. 1045:71-100; Tsuchikama and An, “Antibody-drug conjugates: recent advances in conjugation and linker chemistries”, (2018) Protein & Cell 9(1):33-46; and elsewhere.

Numerous strategies are available for linking the CpG oligonucleotide and targeting moiety or antigen directly, or indirectly via a linker. For example, the CpG oligonucleotide may be derivatized by covalently attaching a linker to the 3′ end region of the CpG oligonucleotide, where the linker has a functional group capable of reacting with a “chemical handle” on the targeting moiety or antigen. Also, by way of example, the targeting moiety or antigen may be derivatized by covalently attaching a linker to the targeting moiety or antigen, where the linker has a functional group capable of reacting with a “chemical handle” on the CpG oligonucleotide. The functional group on the linker may vary and may be selected based on compatibility with the chemical handle on the CpG oligonucleotide or targeting moiety or antigen. According to one embodiment, the chemical handle is provided by incorporation of an unnatural amino acid having the chemical handle into the targeting moiety or antigen. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into a targeting moiety or antigen of a conjugate of the present disclosure, which unnatural amino acid may be selected to provide a functional group of interest, are known and described in, e.g., Maza et al., “Synthesis and Incorporation of Unnatural Amino Acids To Probe and Optimize Protein Bioconjugations”, (2015) Bioconjug. Chem. 26(9):1884-9; Patterson et al., “Finding the right (bioorthogonal) chemistry”, (2014) ACS Chem. Biol. 9:592-605; Adumeau et al., “Site-Specifically Labeled Immunoconjugates for Molecular Imaging-Part 2: Peptide Tags and Unnatural Amino Acids”, (2016) Mol. Imaging Biol. (2):153-65; and elsewhere. An unnatural amino acid may be incorporated into a targeting moiety or antigen via chemical synthesis or recombinant approaches, e.g., using a suitable orthogonal amino acyl tRNA synthetase-tRNA pair for incorporation of the unnatural amino acid during translation of the targeting moiety or antigen in a host cell.

The functional group of an unnatural amino acid present in the targeting moiety or antigen may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the targeting moiety or antigen and the linker portion of a linker-agent moiety may include an alkyne functional group, such that the CpG oligonucleotide or targeting moiety or antigen and linker-agent moiety are covalently conjugated via azide-alkyne cycloaddition. Conjugation may be carried out using, e.g., a copper-catalyzed azide-alkyne cycloaddition reaction, copper-free click chemistry (e.g., by strain-promoted azide-alkyne cycloaddition), or the like.

In certain embodiments, the chemical handle on the targeting moiety or antigen does not involve an unnatural amino acid. A targeting moiety or antigen containing no unnatural amino acids may be conjugated by utilizing, e.g., nucleophilic functional groups of the targeting moiety or antigen (such as the N-terminal amine or the primary amine of lysine, or any other nucleophilic amino acid residue) as a nucleophile in a substitution reaction with a moiety bearing a reactive leaving group or other electrophilic group. An example would be to prepare a targeting moiety- or antigen-linker moiety bearing an N-hydroxysuccinimidyl (NHS) ester and allow it to react with the targeting moiety or antigen under aqueous conditions at elevated pH (~8.5) or in polar organic solvents such as DMSO with an added non-nucleophilic base, such as N,N-diisopropylethylamine.

It will be appreciated that the particular approach for attaching a linker, CpG oligonucleotide and/or targeting moiety or antigen to each other may vary depending upon the particular linker, CpG oligonucleotide and/or targeting moiety or antigen and functional groups selected and employed for conjugating the various components to each other.

### E. Compositions

Aspects of the present disclosure further include compositions comprising a conjugate of the present disclosure. For example, the conjugate may be any of the conjugates described in the Conjugates section above or in the Experimental section below, which descriptions are incorporated but not reiterated herein for purposes of brevity.

In some embodiments, a composition of the present disclosure includes the conjugate present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

A composition of the present disclosure may be suitable for administration to a subject in need thereof. In some embodiments, such compositions comprise a conjugate of the present disclosure, and a pharmaceutically acceptable carrier.

The conjugates can be incorporated into a variety of formulations for therapeutic administration. More particularly, the conjugates can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the conjugates for administration to an individual (e.g., suitable for human administration) are generally sterile and may further be free of detectable pyrogens or other contaminants contraindicated for administration to a patient according to a selected route of administration.

In pharmaceutical dosage forms, the conjugates can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The conjugates can be formulated for parenteral (e.g., intravenous, intra-arterial, intraosseous, intramuscular, intracerebral, intracerebroventricular, intrathecal, subcutaneous, etc.) administration. In certain embodiments, the conjugates are formulated for injection by dissolving, suspending or emulsifying the conjugate in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the conjugates may be prepared by mixing the conjugate having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the conjugates may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™) Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the conjugates against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, a composition includes the conjugate, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% weight/volume (w/v).

### II. Methods of Use

Aspects of the present disclosure further include methods of using the conjugates of the present disclosure. For example, in certain embodiments, provided are methods comprising administering to a subject in need thereof an effective amount of a composition of the present disclosure, e.g., a composition comprising any of the conjugates of the present disclosure.

### A. Methods of Treating Cancer

In certain embodiments, when the conjugate comprises a targeting moiety that binds to an antigen on cancer cells (e.g., a tumor antigen), the method may be a method of treating cancer in a subject in need thereof, the method comprising administering to the subject a composition comprising the conjugate in an amount effective to treat the cancer.

Accordingly, in certain embodiments, the subject has cancer. The methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, cancers comprising a solid tumor, e.g., blastoma, carcinoma, lymphoma, or sarcoma. In certain embodiments, the cancer comprises a hematological malignancy. More particular examples of such cancers include adrenal cancer such as but not limited to, adrenocortical carcinoma and pheochromocytoma; and bladder cancers such as but not limited to, adenocarcinoma, carcinosarcoma, squamous cell cancer, and transitional cell carcinoma; basal cancers; benign monoclonal gammopathy; bone cancer and connective tissue sarcomas such as but not limited to, angiosarcoma (hemangiosarcoma), bone sarcoma, cholesteatoma-induced bone osteosarcoma, chondrosarcoma, chordoma, Ewing's sarcoma, fibrosarcoma, fibrosarcoma of bone, Kaposi's sarcoma, leiomyosarcoma, liposarcoma, lymphangio sarcoma, malignant giant cell tumor, multiple myeloma, myeloma bone disease, neurilemmoma, osteosarcoma, Paget's disease of bone, periosteal sarcoma, rhabdomyosarcoma, soft-tissue sarcomas, and synovial sarcoma; brain tumors such as but not limited to, acoustic neurinoma, astrocytoma, brain stem glioma, craniopharyngioma, ependymoma, glioma, medulloblastoma, meningioma, nonglial tumor, oligodendroglioma, pineoblastoma, pineocytoma, and primary brain lymphoma; breast cancer including but not limited to, adenocarcinoma, inflammatory breast cancer, intraductal carcinoma, lobular (small cell) carcinoma, medullary breast cancer, mucinous breast cancer, Paget's disease (including juvenile Paget's disease), papillary breast cancer, and tubular breast cancer; breast carcinoma; breast sarcoma; carcinomas of the epidermis; cervical cancers such as but not limited to, adenocarcinoma, and squamous cell carcinoma; cervical carcinoma; cholangiocarcinomas such as but not limited to, diffuse, nodular, and papillary; colon cancers; colon carcinoma; colorectal cancer and KRAS mutated colorectal cancer; esophageal cancers such as but not limited to, adenocarcinoma, adenoid cyctic carcinoma, adenosquamous carcinoma, melanoma, mucoepidermoid carcinoma, oat cell (small cell) carcinoma, plasmacytoma, sarcoma, squamous cancer, and verrucous carcinoma; eye cancers such as but not limited to, choroidal melanoma, ciliary body melanoma, ocular melanoma such as iris melanoma, and retinoblastoma; gallbladder cancers such as adenocarcinoma; glioblastoma multiforme; heavy chain disease; kidney cancers such as but not limited to, adenocarcinoma, fibrosarcoma, hypernephroma, renal cell cancer, and transitional cell cancer (renal pelvis and/or uterer); leukemias such as but not limited to, acute leukemia, acute lymphocytic leukemia, acute myelocytic leukemias such as erythroleukemia leukemias, monocytic, myeloblastic, myelodysplastic syndrome, myelomonocytic, promyelocytic, chronic leukemias such as but not limited to hairy cell leukemia, chronic lymphocytic leukemia, and chronic myelocytic (granulocytic) leukemia; liver cancers such as but not limited to, hepatoblastoma and hepatocellular carcinoma; lung cancers such as adenocarcinoma, KRAS-mutated non-small cell lung cancer, large-cell carcinoma, non-small cell lung cancer, small-cell lung cancer and squamous cell carcinoma (epidermoid carcinoma); lung carcinoma; lymphomas such as but not limited to Hodgkin's disease and non-Hodgkin's disease; malignant melanoma; metastatic breast cancer; monoclonal gammopathy of undetermined significance; multiple myelomas such as but not limited to, extramedullary plasmacytoma, nonsecretory myeloma, osteosclerotic myeloma, plasma cell leukemia, smoldering multiple myeloma and solitary plasmacytoma; neuroblastoma; neurofibroma; neurofibromatosis; oral cancers such as but not limited to, squamous cell carcinoma; ovarian cancers such as but not limited to, borderline tumor, germ cell tumor, ovarian epithelial carcinoma, and stromal tumor; pancreatic cancer such as but not limited to, carcinoid or islet cell tumor, gastrinoma, glucagonoma, insulinoma, somatostatin-secreting tumor, and vipoma; pediatric tumors; penal cancers; pharynx cancers such as but not limited to, squamous cell cancer, and verrucous; pituitary cancers such as but limited to acromegaly, Cushing's disease, diabetes insipidus and prolactin-secreting tumors; polycythemia vera; prostate cancers such as but not limited to, rhabdomyosarcoma, seminoma, spermatocytic and teratoma carcinoma; rectal cancers; renal cancer; renal carcinoma; salivary gland cancers such as but not limited to, adenocarcinoma, adenoidcystic carcinoma, and mucoepidermoid carcinoma; skin cancers such as but not limited to, basal cell carcinoma, melanoma, including acrallentiginous melanoma, lentigo malignant melanoma, nodular melanoma, and superficial spreading melanoma, and squamous cell carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; testicular cancers such as but not limited to, adenocarcinoma, anaplastic, androgen dependent prostate cancer, androgen-independent prostate cancer, choriocarcinoma (yolk-sac tumor), classic (typical), embryonal carcinoma, germinal tumor, leiomyosarcoma, and nonseminoma; thyroid cancer such as but not limited to, anaplastic thyroid cancer, medullary thyroid cancer, and papillary or follicular thyroid cancer; uterine cancers such as but not limited to, endometrial carcinoma and uterine sarcoma; vaginal cancers such as adenocarcinoma, melanoma, and squamous cell carcinoma; vulvar cancer such as, adenocarcinoma, basal cell carcinoma, melanoma, Paget's disease, sarcoma, and squamous cell carcinoma; Waldenstrom's macroglobulinemia; and Wilms' tumor. In some embodiments, the cancer is bronchogenic carcinoma, cystadenocarcinoma, endotheliosarcoma, epithelial carcinoma, hemangioblastoma, lymphangioendotheliosarcoma, mesothelioma, myxosarcoma, osteogenic sarcoma, papillary adenocarcinomas, papillary carcinoma, sebaceous gland carcinoma, sweat gland carcinoma or synovioma.

### B. Vaccination Methods

In certain embodiments, when the composition is an immunogenic composition comprising a conjugate comprising an antigen conjugated via a linker to the 3′ end region of a CpG oligonucleotide (e.g., the conjugate may be a vaccine present in an immunogenic composition), the method may be a method of inducing an immune response in a subject, the method comprising administering to the subject an effective amount of the immunogenic composition.

This method may be used to further induce an immune response to a microbial antigen, with a greater response than with the antigen alone. The antigen may be a bacterial antigen, a viral antigen, or a tumor antigen.

### C. Treatment Strategies

By “treat” or “treatment” is meant at least an amelioration of one or more symptoms associated with the condition of the subject (e.g., cancer), where amelioration is used in a broad sense to refer to at least a reduction in the magnitude of a parameter, e.g., symptom, associated with the condition being treated. As such, treatment also includes situations where the condition (e.g., cancer), or at least one or more symptoms associated therewith, are completely inhibited, e.g., prevented from happening, or stopped, e.g., terminated, such that the subject no longer suffers from the condition, or at least the symptoms that characterize the condition.

The compositions of the present disclosure may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, intra-tumoral administration, intraperitoneal (IP) administration, or any other suitable route of administration.

The compositions of the present disclosure may be administered (e.g., in a pharmaceutical composition) in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer, as compared to a control. With respect to cancer, in some embodiments, the therapeutically effective amount is sufficient to slow the growth of a tumor, reduce the size of a tumor, and/or the like. An effective amount can be administered in one or more administrations.

### D. Combination Therapy

In certain embodiments, a composition of the present disclosure is administered to a subject receiving an immune checkpoint inhibitor therapy. In some instances, the immune checkpoint inhibitor therapy comprises administration to the subject of a therapeutically effective amount of an inhibitor of B7-H3, CTLA-4, LAG-3, PD-1, PD-L1, TIGIT, TIM-3, VISTA, or any combination thereof. According to some embodiments, the immune checkpoint inhibitor therapy comprises administration to the subject of a therapeutically effective amount of a PD-1 inhibitor, e.g., an anti-PD1 antibody. In certain embodiments, when the immune checkpoint inhibitor therapy comprises administration to the subject of a therapeutically effective amount of a PD-1 inhibitor, the PD-1 inhibitor is an anti-PD1 antibody, non-limiting examples of which include camrelizumab, cemiplimab, dostarlimab, nivolumab, pembrolizumab, pidilizumab, pimivalimab, retifanlimab, sintilimab, spartalizumab, tislelizumab and toripalimab. According to some embodiments, the immune checkpoint inhibitor therapy comprises administration to the subject of a therapeutically effective amount of a PD-L1 inhibitor, e.g., an anti-PD-L1 antibody. In certain embodiments, when the immune checkpoint inhibitor therapy comprises administration to the subject of a therapeutically effective amount of a PD-L1 inhibitor, the PD-L1 inhibitor is an anti-PD-L1 antibody, non-limiting examples of which include Atezolizumab, Avelumab, and Durvalumab. In embodiments where a composition of the present disclosure is administered to a subject receiving an immune checkpoint inhibitor therapy, the methods may comprise administering the immune checkpoint inhibitor therapy to the subject.

A conjugate of the present disclosure may be administered to the subject alone or in combination with a second agent, e.g., any desired second agent, including but not limited to an immune checkpoint inhibitor as described elsewhere herein. Second agents of interest include, but are not limited to, agents approved by the United States Food and Drug Administration and/or the European Medicines Agency (EMA) for use in treating cancer.

When a conjugate of the present disclosure is administered with a second agent (e.g., an immune checkpoint inhibitor or any other second agent of interest), the conjugate and the second agent may be administered to the individual according to any suitable administration regimen. According to certain embodiments, the conjugate and the second agent are administered according to a dosing regimen approved for individual use. In some embodiments, the administration of the conjugate permits the second agent to be administered according to a dosing regimen that involves one or more lower and/or less frequent doses, and/or a reduced number of cycles as compared with that utilized when the second agent is administered without administration of the conjugate. In certain aspects, the administration of the second agent permits the conjugate to be administered according to a dosing regimen that involves one or more lower and/or less frequent doses, and/or a reduced number of cycles as compared with that utilized when the conjugate is administered without administration of the second agent.

In some embodiments, one or more doses of the conjugate and the second agent are administered concurrently to the individual. By “concurrently” is meant the conjugate and the second agent are either present in the same pharmaceutical composition, or the conjugate and the second agent are administered as separate pharmaceutical compositions within 1 hour or less, 30 minutes or less, or 15 minutes or less.

In some embodiments, one or more doses of the conjugate and the second agent are administered sequentially to the individual.

In some embodiments, the conjugate and the second agent are administered to the individual in different compositions and/or at different times. For example, the conjugate may be administered prior to administration of the second agent, e.g., in a particular cycle. Alternatively, the second agent may be administered prior to administration of the conjugate, e.g., in a particular cycle. The second agent to be administered may be administered over a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the conjugate. In certain aspects, when the individual has cancer, such a regimen “primes” the cancer cells to potentiate the anti-cancer effect of the conjugate. Such a period of time separating a step of administering the second agent from a step of administering the conjugate is of sufficient length to permit priming of the cancer cells, desirably so that the anti-cancer effect of the conjugate is increased.

In some embodiments, administration of one agent is specifically timed relative to administration of the other agent. For example, in some embodiments, the conjugate is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

In some embodiments, the conjugate and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the conjugate and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the conjugate is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the conjugate is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered; properties (e.g., pharmacokinetic properties) of the conjugate, and/or one or more features of the patient's response to therapy with the conjugate. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The conjugate and the second agent may be administered together or independently via any suitable route of administration. The conjugate and the second agent may be administered via a route of administration independently selected from oral, parenteral (e.g., by intravenous, intra-arterial, subcutaneous, intramuscular, or epidural injection), topical, or intra-nasal administration. According to certain embodiments, the conjugate and the second agent are both administered orally (e.g., in tablet form, capsule form, liquid form, or the like) either concurrently (in the same pharmaceutical composition or separate pharmaceutical compositions) or sequentially.

The following examples are offered by way of illustration and not by way of limitation.

### EXPERIMENTAL EXAMPLES

### Example 1—Chemically Modifying the 5′ and 3′ End Regions of CpG

### Rationale for PIP-CpG Conjugates as Model System:

Prior work disclosed a tumor-targeting peptide-CpG conjugate, where a tumor-targeting peptide, called PIP, was conjugated to the 5′ end of a particular Class-C CpG oligonucleotide, CpG-C SD101 (SEQ NO: 1), resulting in a conjugate herein referred to as 5′ PIP-CpG (CpG-C SD101).^{1,6} This conjugate model system of attaching CpG oligonucleotides to a tumor-targeting PIP peptide (i.e., PIP-CpG conjugates) was used as proof of principle examples throughout this work for the following reasons:

First, these PIP-CpG conjugates served as useful examples that have potential for therapeutic applications. The previously disclosed 5′ PIP-CpG (CpG-C SD101)^{1,6} conjugate demonstrated improved therapeutic efficacy relative to unmodified CpG when delivered intravenously in multiple mouse cancer models.^{1,6} Importantly, this improved efficacy with 5′ PIP-CpG (CpG-C SD101) was due to enhanced tumor delivery in vivo. However, when evaluated through in vitro immune-cell based assays, PIP-CpG conjugates are expected to have similar potency as the unmodified CpG since the tumor-targeting mechanism is not particularly relevant for these in vitro immune assays. Since the goal of this series of experiments was to evaluate optimal conjugation design principles for class C CpG oligonucleotides, using PIP-CpG conjugates as a model system was ideal for assessment through in vitro immune cell-based assays, enabling head-to-head comparisons between conjugates and unmodified CpG conjugates.

Second, using PIP-CpG conjugates as a model system enabled homogeneous, site-specific conjugation chemistry to be used, reducing complexity and variation between the CpG conjugates described herein. The simplicity of the design minimizes potential confounding variables and enables stronger support for the drawn conclusions. The PIP-CpG conjugates described in this series of experiments all consist of exactly 1 PIP peptide per CpG oligonucleotide.

### Chemically Modified CpG Oligonucleotides:

Dibenzocyclooctyne (DBCO)-modified CpG oligonucleotides used throughout these series of experiments were purchased from vendors, such as Integrated DNA Technologies, BioSpring, CellMosaic, and WuXi STA. For clarity, the general conjugation scheme that was used by vendors to functionalize CpG oligonucleotides with DBCO is described briefly here—In order to generate DBCO-modified CpG conjugates, CpG oligonucleotides were first synthesized to incorporate either a 5′ Amino-C6 modifier or a 3′ Amino-C6 modifier to produce 5′ Amino-CpG or 3′ Amino-CpG, respectively. The 5′ Amino-CpG and 3′ Amino-CpG were then further modified to incorporate a dibenzocyclooctyne (DBCO) group using DBCO-PEG4-NHS ester to produce 5′ DBCO-CpG or 3′ DBCO-CpG, respectively. Unmodified CpG oligonucleotides used throughout these experiments were purchased from vendors, such as Invivogen, the Stanford Protein and Nucleic Acid Facility (PAN), and Integrated DNA Technologies.

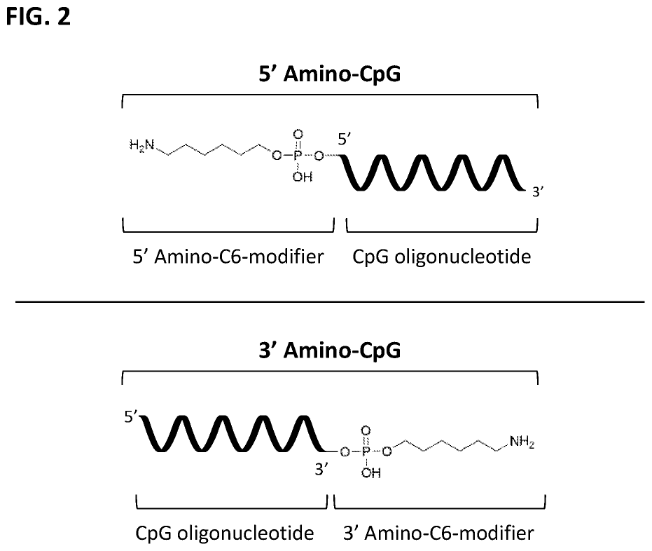

FIG. 2 shows the general structure of 5′ Amino-CpG and 3′ Amino-CpG oligonucleotides.

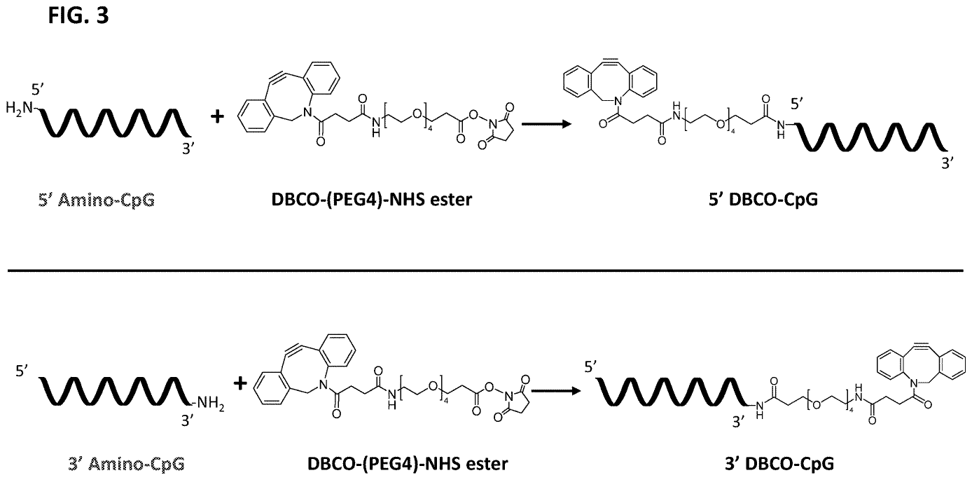

FIG. 3 shows the synthesis strategy for producing 5′ DBCO-CpG and 3′ DBCO-CpG.

### Synthesis of PIP-CpG Conjugates

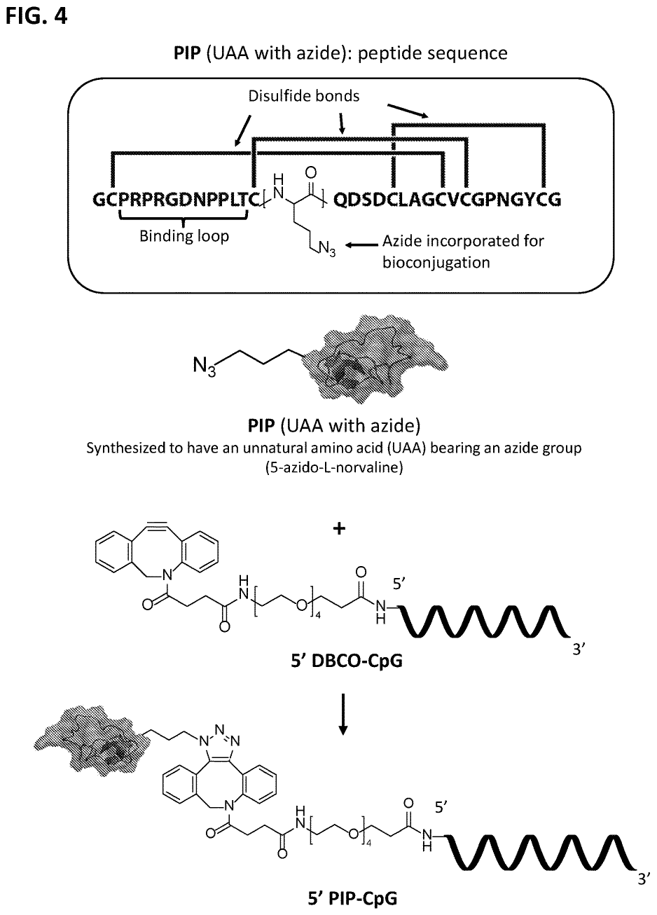

FIG. 4 shows the 5′ PIP-CpG conjugation strategy. This is the general conjugation scheme that was used in previously published work with the 5′ PIP-CpG (CpG-C SD101) conjugate.^{1,6} The same conjugation strategy was used to generate 5′ PIP-CpG conjugates in this series of experiments. The PIP peptide, made via solid phase peptide synthesis, contains an unnatural amino acid, 5-azido-L-norvaline, at position 15 in the peptide sequence (SEQ ID NO: 18), and was purchased from CSBio for this series of experiments. The synthesis of 5′ PIP-CpG was performed as previously described^{1,6} with some modifications. Briefly, to produce the 5′ PIP-CpG conjugate, 5′ DBCO-CpG was mixed with 1.1 eq PIP (which contains an unnatural amino acid bearing an azide group) in PBS at 30° C. on a thermomixer at 500 rpm overnight (18-24 h) in the dark. The 5′ PIP-CpG conjugate was purified from unreacted PIP peptide using Zeba spin desalting columns (7K MWCO) that were buffer exchanged into PBS prior to sample loading. Analytical ion-pair reverse-phase HPLC was used to monitor the reaction and to assess the final purified product. The same methods were applied to produce the 3′ PIP-CpG conjugate with the exception that 3′ DBCO-CpG was used in place of 5′ DBCO-CpG.

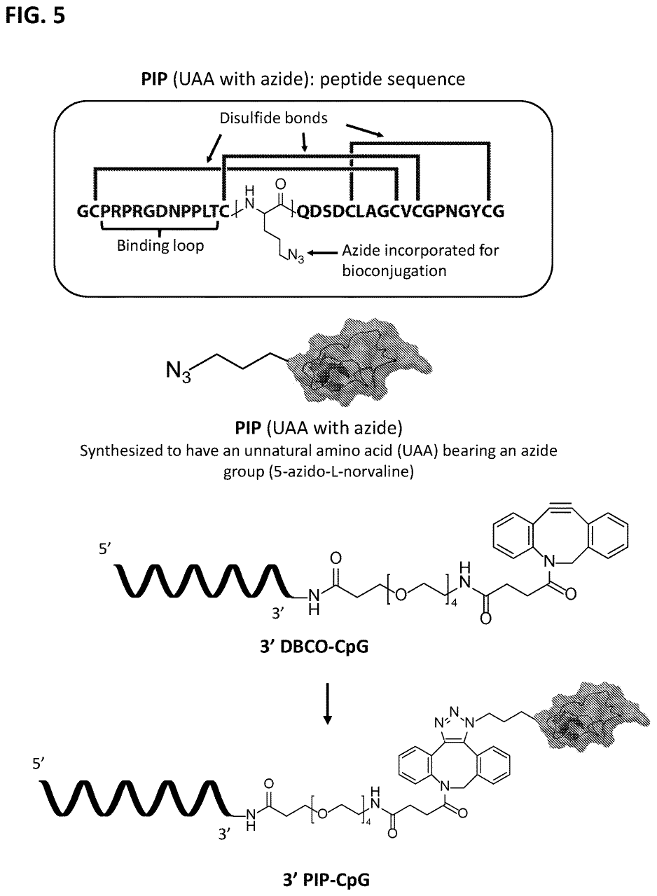

FIG. 5 shows the 3′ PIP-CpG conjugation strategy.

### Example 2—Class-C CpG Conjugation Site can Selectively Modulate IFNα Production

### Human PBMC Stimulation Assays

Human peripheral blood mononuclear cells (PBMCs) were isolated using Ficoll-Paque density gradient separation and were then used immediately for stimulation assays (not cryopreserved). PBMCs (600,000 cells/well) were incubated with CpG conjugates and relevant controls in 96-well round-bottom plates at 37° C. in 5% CO₂ for 24 h. RPMI+10% HI FBS+1% P/S was used as the culture media for incubations (200 μL/well final volume). After the 24 h incubation, the supernatant was collected and stored frozen at −20° C. On a separate day, the supernatant was thawed for assessment of IFNα or proinflammatory cytokine (i.e., IL-6, TNF, IL-1β, IL-10) levels. To assess IFNα levels, the supernatant was analyzed using the IFN alpha (Multisubtype) Human ELISA Kit (Invitrogen: 411051). To assess proinflammatory cytokine levels, the supernatant was analyzed using the BD Cytometric Bead Array (CBA) Human Inflammatory Cytokine Kit (BD Biosciences: 551811) on a BD Accuri C6 Plus flow cytometer. Error bars represent the SD of 3 replicates.

### RAW-Blue Activation Assays

CpG conjugates and relevant controls were evaluated using the RAW-Blue NF-κB-SEAP Reporter Cell Line (Invivogen, raw-sp), derived from murine RAW 264.7 macrophages. RAW-Blue cells (100,000 cells/well) were incubated with CpG conjugates and relevant controls at various concentrations in 200 μL/well in 96-well flat-bottom plates for 22-24 h at 37° C. in 5% CO₂ in DMEM+10% HI FBS+1% P/S. After incubation, the levels of SEAP in the supernatant were assessed as specified in the manufacturer's protocol. Error bars represent the SD of 3 replicates. Data are reported as fold change in NF-κB/AP-1 activity compared to the untreated control (value of sample divided by the average value of untreated replicates).

### 5′ Class C CpG Conjugates Retain TLR9-Mediated NF-κB Activation Capacity:

In previously published work, the 5′ PIP-CpG (CpG-C SD101) conjugate showed similar potency as the unmodified class C CpG (CpG-C SD101) in a RAW-Blue activation assay, which measures NF-κB activation.^{1,6} This published data suggested that the conjugation of PIP to the 5′ end of the Class-C CpG SD101 did not compromise TLR9 agonist potency, at least in this particular RAW-Blue NF-κB activation assay.^{1,6}

In this series of experiments, 5′ Amino-CpG (CpG-C SD101) was also tested in the RAW-Blue assay to verify the smaller 5′ amino modification would also retain NF-κB activation capacity.

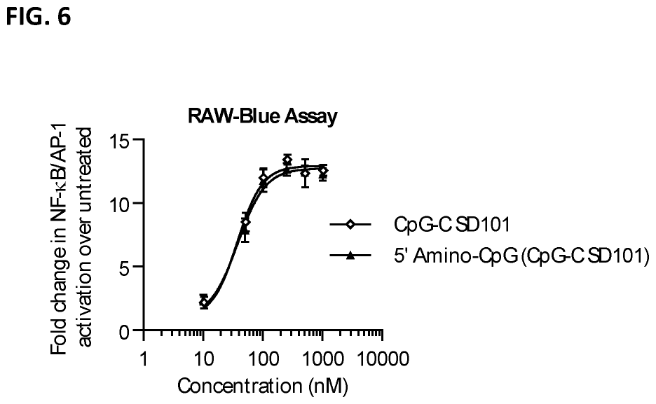

FIG. 6 shows that 5′ Amino-CpG (CpG-C SD101) retains full NF-κB activation capacity relative to the corresponding unmodified CpG (CpG-C SD101) in the RAW-Blue NF-κB activation assay.

In this series of experiments, to further verify that the 5′ PIP-CpG (CpG-C SD101) fully retained its TLR9-mediated NF-κB activation relative to unmodified CpG, 5′ PIP-CpG (CpG-C SD101) was tested in comparison to CpG-C SD101 in human PBMC stimulation assays that measured production of inflammatory cytokines that are associated with TLR9-mediated NF-κB activation (e.g., IL-6, TNF, IL-1β, IL-10).

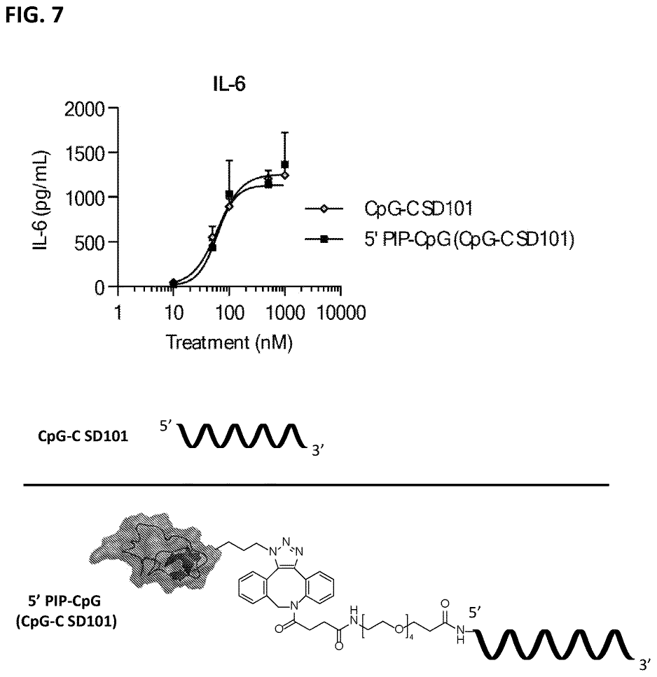

FIG. 7 shows that 5′ PIP-CpG (CpG-C SD101) has a similar capacity as CpG-C SD101 to stimulate production of IL-6 in human PBMCs.

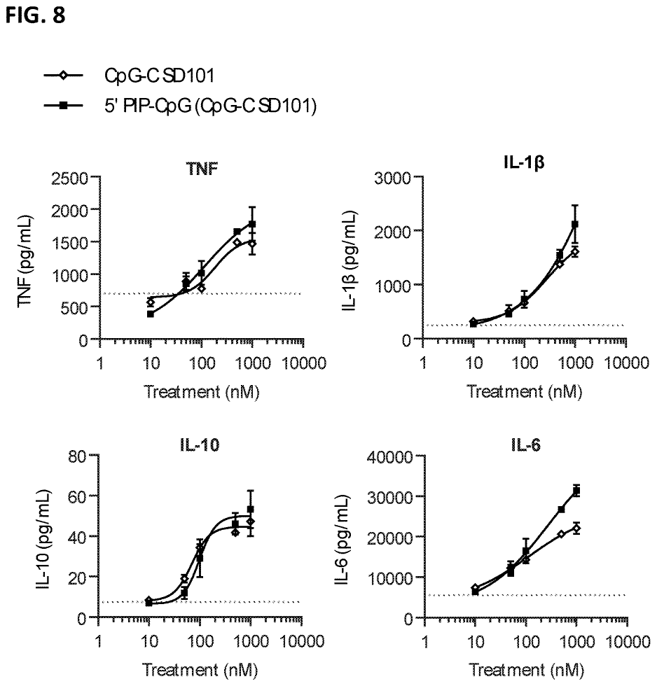

FIG. 8 shows that 5′ PIP-CpG (CpG-C SD101) has a similar capacity as CpG-C SD101 to stimulate production of TNF, IL-1, IL-10, and IL-6 in human PBMCs. The dashed lines represent the average levels of these cytokines produced by untreated cells in this particular experiment.

These results add additional support to the previously published findings^{1,6} that the Class C CpG oligonucleotide, CpG-C SD101, can be modified at the 5′ end of the CpG without compromising TLR9-mediated NF-κB activation.

### 5′ Class C CpG Conjugates Show Diminished TLR9-Mediated IFNα Production:

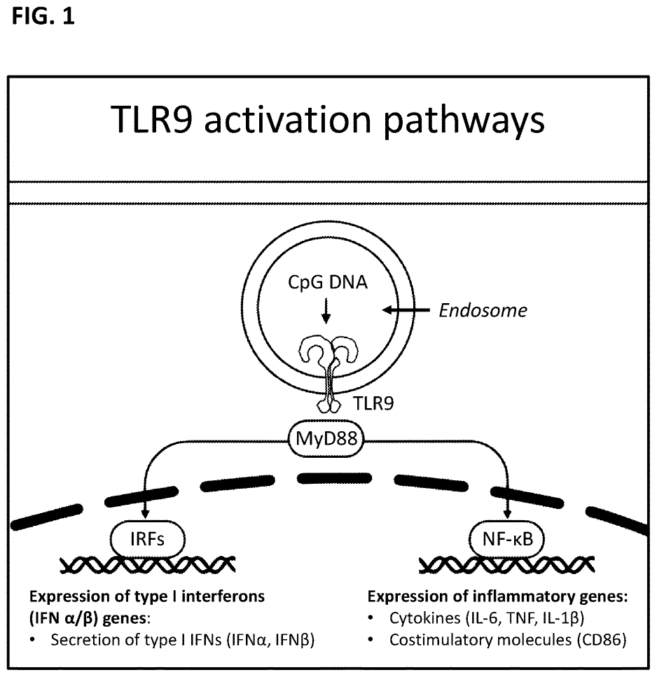

Class C CpG binding to TLR9 can lead to robust activation of two downstream signaling pathways: NF-κB and interferon regulatory factor (IRF) pathways (FIG. 1). Activation of the NF-κB pathway leads to the induction of proinflammatory cytokines (e.g., IL-6, TNF-α) as well as prominent B cell activation and proliferation, whereas activation of the IRF pathway (particularly IRF7) primarily leads to the production of type I interferons (e.g., IFNα).

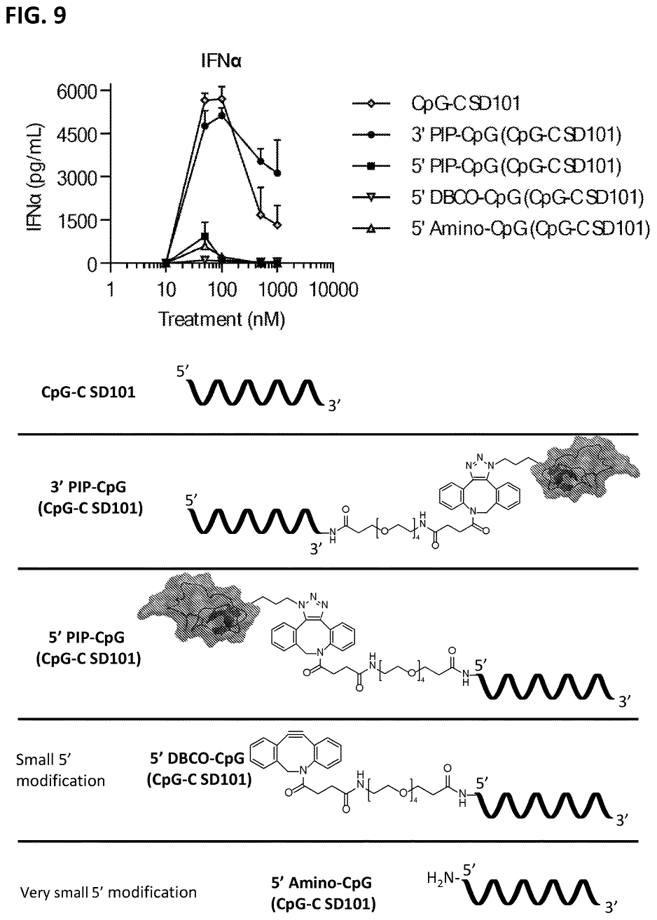

FIG. 9 shows that chemical modification at the 5′ end of CpG-C SD101 selectively impairs IFNα production in human PBMCs relative to corresponding unmodified CpG. Three 5′ modified CpG-C SD101 conjugates (5′ PIP-CpG, 5′ DBCO-CpG, and 5′ Amino-CpG), which vary in size and chemical properties, are shown in this experiment to demonstrate the findings are broad and observed for multiple chemical conjugates.

### Conjugation at the 3′ End Regions of a Class C CpG Restores Full Capacity for Both NF-κB-Based Activation and IFNα Production

FIG. 9 also shows that, in contrast to the 5′ CpG conjugates, 3′ PIP-CpG (CpG-C SD101) was able to stimulate similar amounts of IFNα production as the corresponding unmodified CpG in human PBMCs.

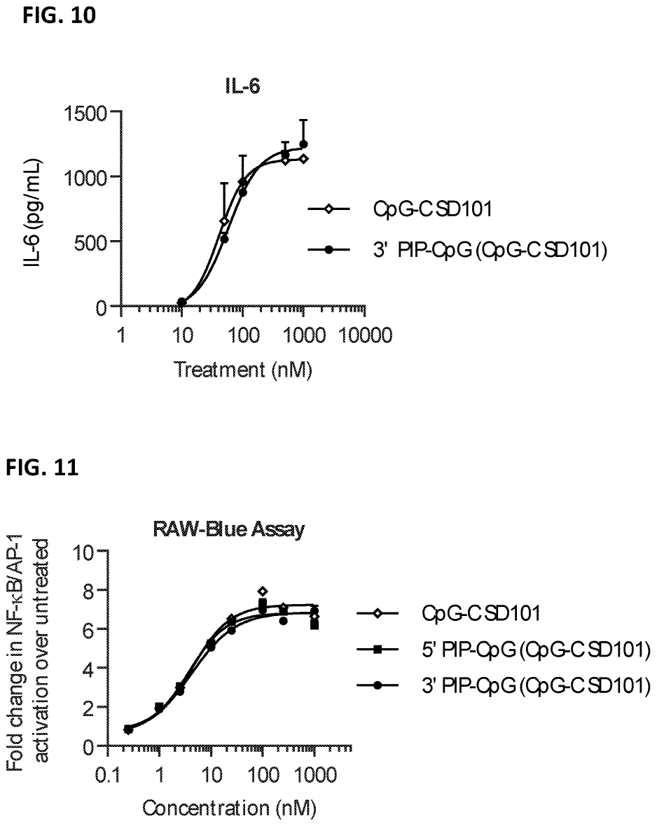

FIG. 10 shows that 3′ PIP-CpG (CpG-C SD101) has a similar capacity to stimulate the NF-κB activation pathway as the corresponding unmodified CpG (CpG-C SD101) as measured by the production of IL-6 in human PBMCs.

FIG. 11 shows that both the 5′ PIP-CpG (CpG-C SD101) and 3′ PIP-CpG (CpG-C SD101) have similar capacities to stimulate the NF-κB activation pathway as the corresponding unmodified CpG in the RAW-Blue activation assay.

Collectively, these data demonstrate that 3′ PIP-CpG retains full capacity for both NF-κB-based activation and IFNα production, whereas 5′ Class C CpG conjugates retain full NF-κB-based activation but exhibit minimal IFNα production, suggesting the IRF pathway is selectively impaired.

### Example 3—Findings are Unique to Class C CpG Conjugates and are not Observed for Class a or Class B Conjugates

Class C CpG oligonucleotides represent one of the three main classes of CpG oligonucleotides. The other two classes are class A and class B CpG oligonucleotides. These CpG classes are all TLR9 agonists, but they have different sequences and structural properties, which modulate their immunological activity. Class A CpG oligonucleotides can robustly activate IRF signaling, leading to strong induction of IFNα, but they are weak stimulators of NF-κB signaling and its associated inflammatory cytokine production (e.g., IL-6). In contrast, class B CpG oligonucleotides show the opposite effect—they strongly activate NF-κB signaling and promote robust inflammatory cytokine production, but they are weak stimulators of IRF signaling, resulting in little or no IFNα production. Class C CpG oligonucleotides are capable of activating both NF-κB and IRF pathways, leading to robust induction of proinflammatory cytokines (e.g., IL-6) and IFNα, respectively.

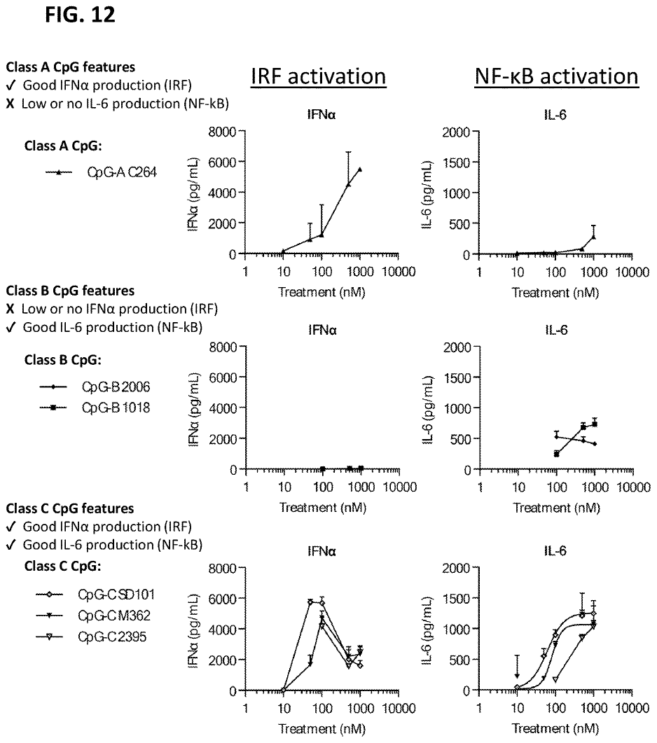

FIG. 12 demonstrates how the three main classes of CpG oligonucleotides have different immunological activities in human PBMC stimulation assays. The example Class A CpG, CpG-A C264 (SEQ ID NO: 12), shows robust IFNα production with much lower IL-6 production. The example Class B CpG oligonucleotides, CpG-B 2006 (SEQ ID NO: 8) and CpG-B 1018 (SEQ ID NO: 9), both show IL-6 production at 100 nM, 500 nM, and 1000 nM, but essentially no IFNα production at any of those concentrations. The example Class C CpG oligonucleotides, CpG-C SD101 (SEQ ID NO: 1), CpG-C M362 (SEQ ID NO: 2), and CpG-C 2395 (SEQ ID NO: 3), show robust IFNα and IL-6 production. Notably, while TLR9-mediated NF-κB readouts like IL-6 usually follow a typical S-shaped dose-response curve, IFNα production from Class-C CpG oligonucleotides often results in a bell-shaped dose response curve.

In the previous example, 5′ Class C CpG conjugates were shown to retain full NF-κB-based activation but demonstrated impaired IFNα production. To determine if conjugation at the 5′ end of CpG would impair TLR9 agonist activity in the other classes of CpG, new 5′ PIP-CpG conjugates were generated using CpG-A C264 (SEQ ID NO: 12) and CpG-B 2006 (SEQ ID NO: 8) sequences.

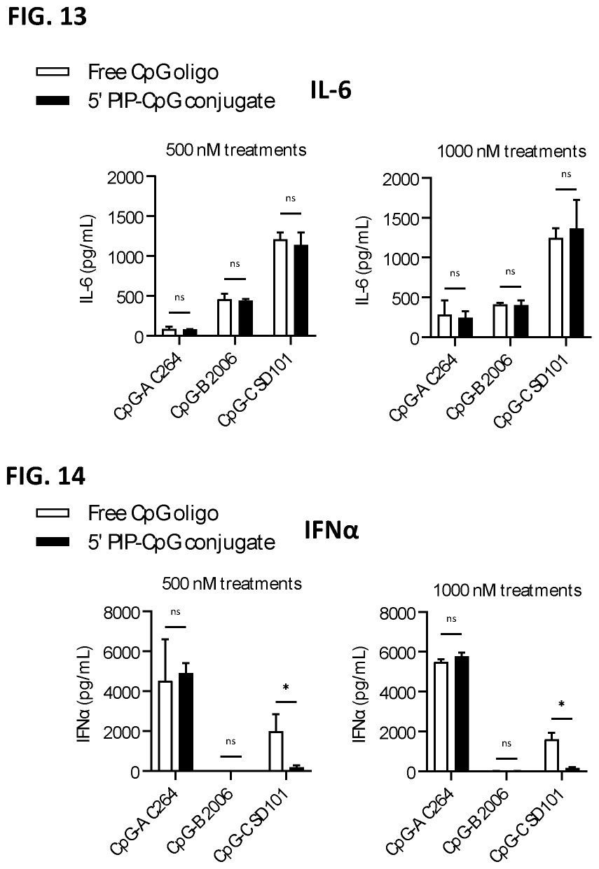

FIG. 13 shows that 5′ PIP-CpG conjugates made with any of the three CpG classes (A, B, C) showed similar levels of IL-6 production relative to their corresponding unmodified CpG control in a human PBMC stimulation assay. Unpaired two-tailed t test. ns indicates not significant (p>0.05).

FIG. 14 shows IFNα production from 5′ PIP-CpG conjugates made with three different CpG classes (A, B, C) in a human PBMC stimulation assay. Given class-B CpG oligonucleotides are not strong inducers of IFNα, neither the 5′ PIP-CpG (CpG-B 2006) or its associated free CpG-B 2006 showed any IFNα production, as expected. In agreement with the unexpected findings shown in Example 2, the 5′ PIP-CpG (CpG-C SD101) conjugate again showed impaired IFNα production relative to the free CpG-C SD101. Surprisingly, in contrast to the Class C CpG conjugate, the 5′ PIP-CpG (CpG-A C264) retained full IFNα production potency relative to its free CpG-A C264 control. Unpaired two-tailed t test. ns indicates not significant (p≥0.05). * indicates p<0.05.

Taken together, these results indicate the unexpected findings (impaired IFNα production with 5′ conjugation) is unique to Class C CpG oligonucleotides. This also means that having a free 5′ end of a CpG oligonucleotide is not a universal requirement for TLR9-mediated IFNα production because the 5′ CpG conjugate based on a Class A CpG (CpG-A C264) was still able to robustly stimulate IFNα production. Furthermore, 5′ CpG conjugates based on class A or Class B CpG oligonucleotides were fully functional, retaining their full TLR9 agonist activity of their associated pathways (i.e., mainly NF-κB for class B; mainly IRF for class A). In contrast, 5′ class-C CpG conjugates only retain full NF-κB pathway activation but have diminished IFNα production.

### Example 4—Findings are Consistent Across Different Class C CpG Sequences

The previous examples used the CpG-C SD101 sequence to generate proof of principle Class C CpG conjugates. To demonstrate the findings are applicable to other class C CpG sequences with different features, a new 5′ PIP-CpG conjugate was generated using CpG-C M362 (SEQ ID NO: 2), herein referred to as 5′ PIP-(CpG-C M362). For the CpG-C SD101 sequence, almost the entire sequence is palindromic (28 of 30 nucleotides), and the palindromic sequence begins at the 5′ end. In contrast, for the newly selected CpG-C M362 sequence, only a portion of the sequence is palindromic (16 of 25 nucleotides) and the palindromic region is located near the middle of the sequence, meaning that the 5′ end is not part of the palindromic region.

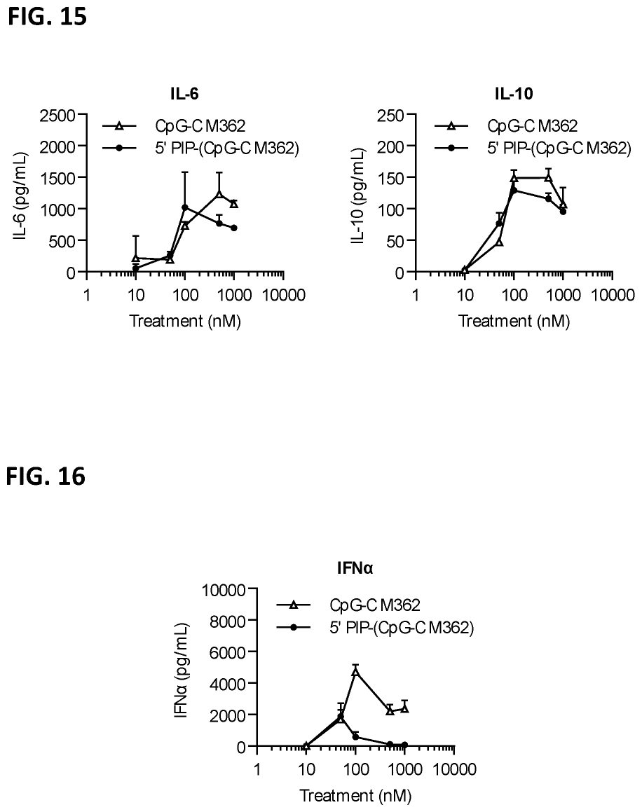

FIG. 15 shows that the 5′ PIP-(CpG-C M362) conjugate had similar capacity as CpG-C M362 to stimulate production of IL-6 and IL-10 in human PBMCs, demonstrating that the conjugate retained NF-κB activation potency.

FIG. 16 shows that the 5′ PIP-(CpG-C M362) conjugate had impaired IFNα production relative to CpG-C M362 in human PBMCs, which is in accordance with the results found for 5′ CpG-C SD101 conjugates.

Taken together, these findings demonstrate that this surprising observation of retained NF-κB potency coupled with impaired IRF activation with 5′ conjugates is observed across structurally different class C CpG sequences.

### Example 5—Findings are Applicable to Different Types of Linkers and are not Dependent on Linker Stability

In the previous examples, the CpG conjugates were all generated using CpG oligonucleotides that were originally synthesized with either a 5′ Amino-C6-modifier or a 3′ Amino-C6-modifier, as shown in FIG. 2. Those amino-modified CpG oligonucleotides were then functionalized with DBCO, which allowed them to be conjugated to PIP. While the 5′ Amino-C6 and 3′ Amino-C6 modifications are known to be quite resistant to exonucleases, it is still possible for oligonucleotides bearing these modifications to get cleaved by exonucleases slowly over time.

For the PIP-CpG conjugates described in the previous examples, the linkers were designed to be stable. However, given that the amino-C6-modifier portion has the potential to be cleaved by exonucleases slowly over time, cleavage resulting in the release of free CpG was still a possibility. Thus, studies were conducted to determine if differences in linker stability could explain the unexpected immunological effects observed for 5′ vs. 3′ Class C CpG conjugates.

First, a new 3′ conjugate, referred to as 3′ PIP-PS-CpG (CpG-C SD101), was generated that was expected to be even more stable than the original 3′ PIP-CpG (CpG-C SD101). The only difference between the two conjugates is that for the 3′ PIP-PS-CpG conjugate, the CpG oligonucleotide was synthesized with a 3′ Amino-C6-PS modifier, which is more stable than the 3′ Amino-C6-modifier used in the 3′ PIP-CpG conjugate.

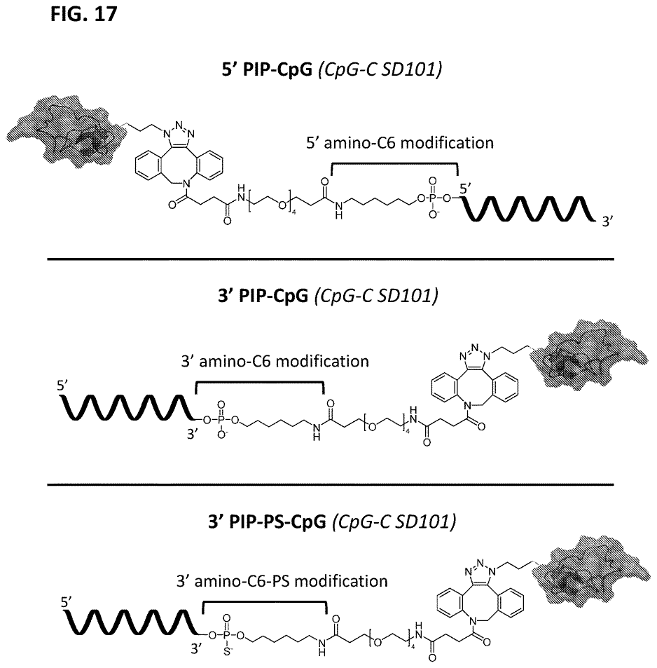

FIG. 17 shows the structure of the new conjugate, 3′ PIP-PS-CpG (CpG-C SD101), relative to 5′ PIP-CpG (CpG-C SD101) and 3′ PIP-CpG (CpG-C SD101) conjugates used in previous examples.

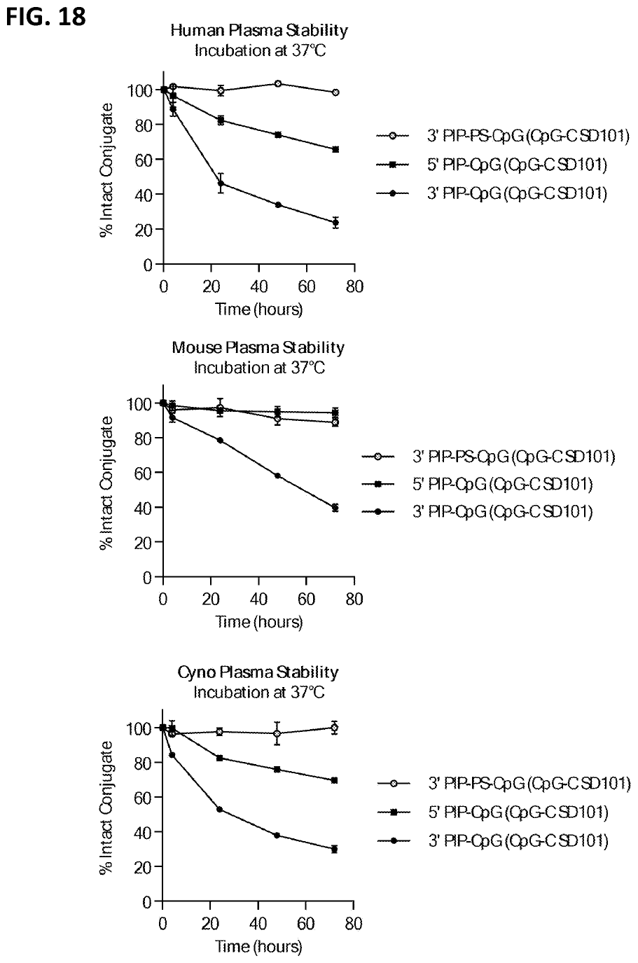

FIG. 18 shows plasma stability over 72 hours of three CpG-C SD101 conjugates: 3′ PIP-PS-CpG, 5′ PIP-CpG, and 3′ PIP-CpG across three species: human, mouse, and cynomolgus monkey. The 3′ PIP-CpG conjugate was susceptible to cleavage in all three species over time, whereas the 3′ PIP-PS-CpG conjugate was highly stable for 72 hours in all three species. The 5′ PIP-CpG conjugate was somewhat susceptible to cleavage in human and cynomolgus monkey plasma, but it showed higher stability than the 3′ PIP-CpG conjugate; in mouse plasma, 5′ PIP-CpG was very stable over 72 hours. It should be noted that, in vivo, these particular PIP-CpG conjugates are expected to clear from blood circulation within a few hours, so stability in plasma for up to 72 hours is not a requirement. Nonetheless, these results demonstrate that the newly generated 3′ PIP-PS-CpG conjugate is indeed much more stable than the original 3′ PIP-CpG conjugate in all three species as well as the 5′ PIP-CpG conjugate in human and cynomolgus monkey plasma.

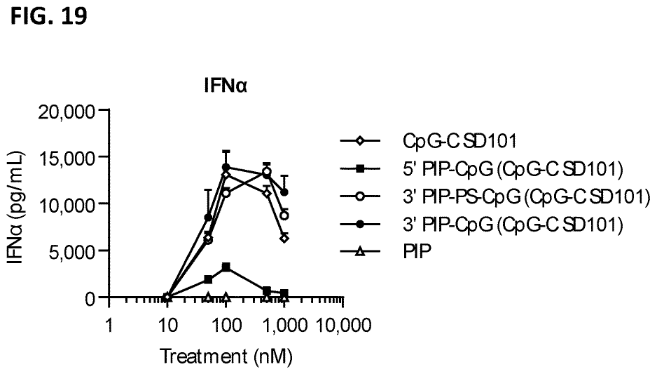

FIG. 19 shows that both 3′ PIP-PS-CpG (CpG-C SD1101) and 3′ PIP-CpG (CpG-C SD101) can stimulate similar amounts of IFNα production as the corresponding unmodified CpG-C SD101 in human PBMCs, whereas 5′ PIP-CpG (CpG-C SD101) again showed diminished capacity for IFNα production. The free PIP peptide was shown as an additional control in this study, and as expected, PIP did not stimulate any IFNα production.

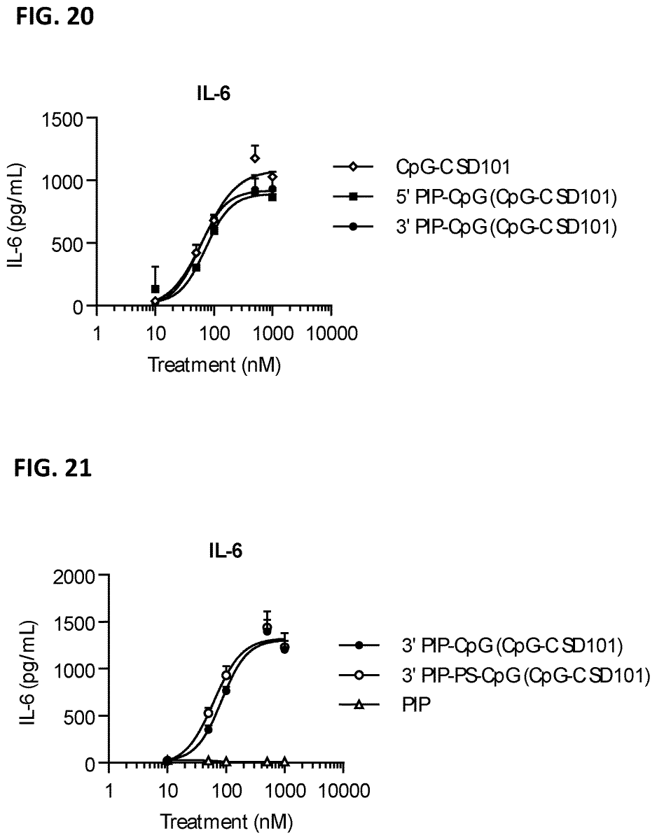

FIG. 20 shows that 5′ PIP-CpG (CpG-C SD101) and 3′ PIP-CpG (CpG-C SD101) had similar capacity as CpG-C SD101 to stimulate IL-6 production.

FIG. 21 shows that 3′ PIP-PS-CpG (CpG-C SD101) had similar capacity as 3′ PIP-CpG (CpG-C SD101) to stimulate IL-6 production. The free PIP peptide was shown as an additional control in this study, and as expected, PIP did not stimulate any IL-6 production.

Taken together, these results show that differences in linker stability do not explain the unexpected immunological effects observed for 5′ vs. 3′ Class C CpG conjugates in previous examples. Therefore, the conclusion of the 3′ end region being optimal for class C CpG conjugation is applicable to different types of linkers, regardless of their stability.

### Example 6—Additional 3′ Class C CpG Conjugates Retain Full TLR9 Agonist Capacity

Given that the 3′ PIP-CpG conjugate retained both NF-κB-based activation and IFNα production, additional 3′ CpG conjugates were synthesized and tested to determine if the findings would be observed for multiple chemical conjugates.

New tumor-targeted CpG conjugates that incorporate MMP2/MMP9-cleavable peptide linkers were synthesized. After localizing to the tumor through the tumor-targeting domain (e.g., PIP), these MMP-cleavable conjugates will allow CpG to be released in the tumor microenvironment, which could facilitate uptake by surrounding immune cells.

### Synthesis of 3′ MMPa-CpG and 3′ MMPb-CpG

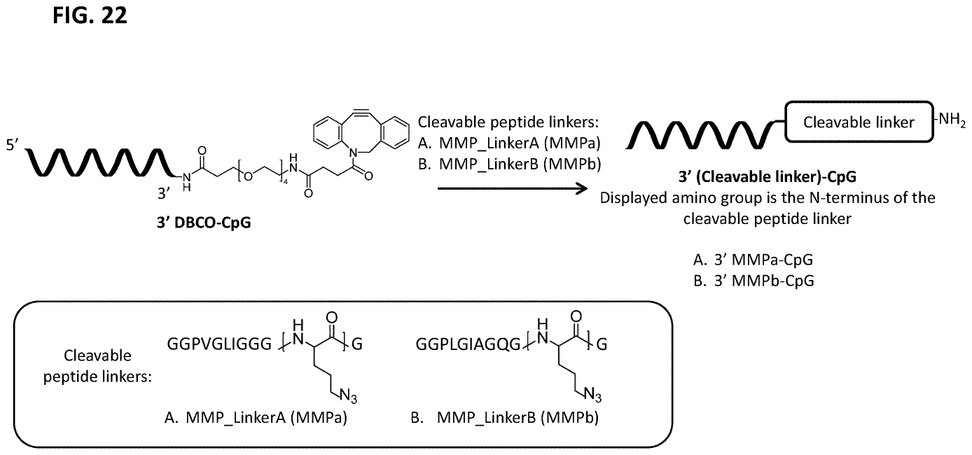

FIG. 22 shows the synthesis strategy for producing 3′ MMPa-CpG and 3′ MMPb-CpG.

Two MMP2/9-cleavable peptide linkers, referred to as MMPa and MMPb, were synthesized by SPPS by the Protein and Nucleic Acid (PAN) facility at Stanford University; an unnatural amino acid, 5-azido-L-norvaline, was incorporated in both cleavable peptide linkers as shown in FIG. 22 to enable click chemistry conjugation.

To produce 3′ MMPa-CpG and 3′ MMPb-CpG, 3′ DBCO-CpG (CpG-C SD101), which was dissolved in PBS, was reacted with 1.2 eq of cleavable peptide linker (MMPa or MMPb; dissolved in DMSO) at 30° C. in a mixture of 85% PBS/15% DMSO on a thermomixer at 500 rpm overnight (18-24 h) in the dark. The 3′ cleavable peptide linker-CpG conjugates were purified from unreacted cleavable peptide linkers using Zeba spin desalting columns (7K MWCO) that were buffer exchanged into PBS prior to sample loading. Analytical ion-pair reverse-phase HPLC was used to monitor reactions and to assess final purified products.

### Synthesis of 3′ DBCO-MMPa-CpG and 3′ DBCO-MMPb-CpG

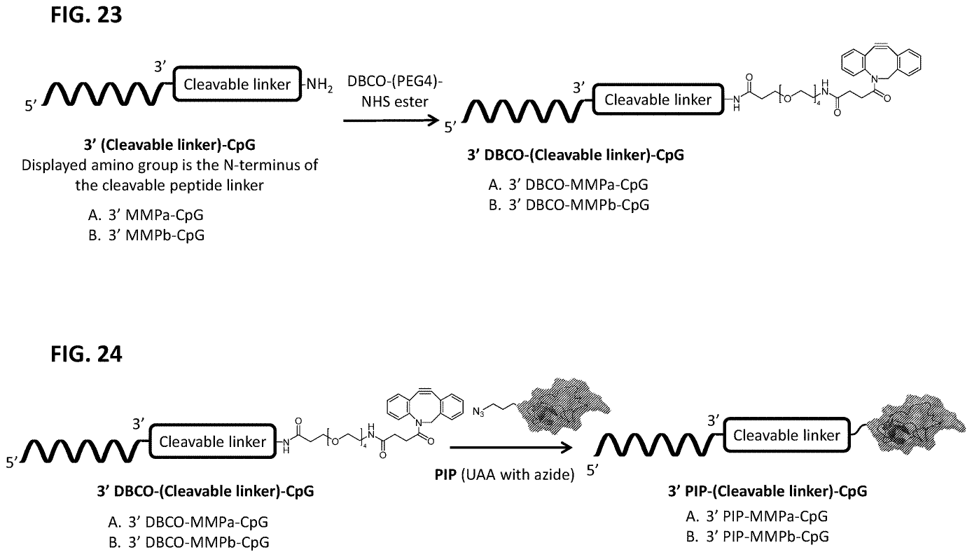

FIG. 23 shows the synthesis strategy for producing 3′ DBCO-MMPa-CpG and 3′ DBCO-MMPb-CpG.

To produce 3′ DBCO-MMPa-CpG and 3′ DBCO-MMPb-CpG, 3′ Cleavable peptide linker-CpG conjugates (i.e., 3′ MMPa-CpG and 3′ MMPb-CpG) were reacted with 15 eq of DBCO-PEG4-NHS ester (dissolved in DMSO) at ambient temperature in a mixture of 65% 100 mM sodium borate buffer (pH 8.5)/35% DMSO on a thermomixer at 500 rpm for 2 h. The products were immediately purified from unreacted DBCO-PEG4-NHS ester and other low molecular weight impurities via two passes through Zeba spin desalting columns (7K MWCO) that were buffer exchanged into PBS prior to sample loading. Analytical ion-pair reverse-phase HPLC was used to monitor reactions and to assess final purified products.

### Synthesis of 3′ PIP-MMPa-CpG and 3′ PIP-MMPb-CpG

FIG. 24 shows the synthesis strategy for producing 3′ PIP-MMPa-CpG and 3′ PIP-MMPb-CpG.

To produce 3′ PIP-MMPa-CpG and 3′ PIP-MMPb-CpG, 3′ DBCO-(cleavable peptide linker)-CpG conjugates (i.e., 3′ DBCO-MMPa-CpG and 3′ DBCO-MMPb-CpG) were reacted with 1.2 eq of PIP (which contains an unnatural amino acid bearing an azide group) in PBS at 30° C. on a thermomixer at 500 rpm overnight (18-24 h) in the dark. The conjugate products were purified from unreacted PIP peptide using Zeba spin desalting columns (7K MWCO) that were buffer exchanged into PBS prior to sample loading. Analytical ion-pair reverse-phase HPLC was used to monitor reactions and to assess final purified products.

### Additional 3′ Class C CpG Conjugates Induce Robust IFNα Production and NF-κB Activation:

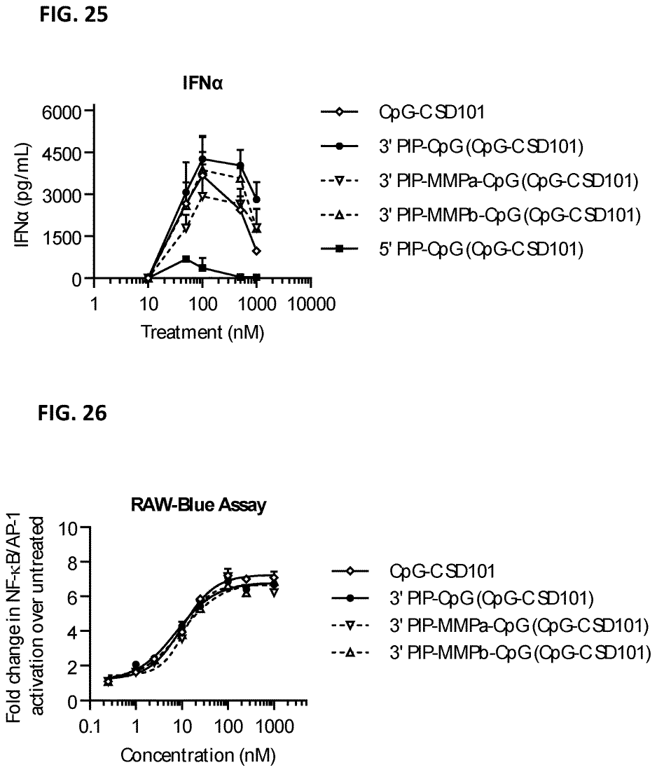

FIG. 25 shows that various 3′ modified CpG conjugates with cleavable linkers, (3′ PIP-MMPa-CpG and 3′ PIP-MMPb-CpG) can stimulate similar amounts of IFNα production as the corresponding unmodified CpG-C SD101 in human PBMCs.

FIG. 26 shows that various 3′ modified CpG conjugates with cleavable linkers (3′ PIP-MMPa-CpG and 3′ PIP-MMPb-CpG) retain full NF-κB activation capacity relative to CpG-C SD101 in the RAW-Blue NF-κB activation assay.

### Example 7—Additional 3′ Class C CpG Conjugates Demonstrate Function Enzymatically-Cleavable Linkers

### MMP2/9 Enzyme Cleavage Assays:

Human MMP2 (R&D Systems: 902-MP-010), mouse MMP2 (R&D Systems: 924-MP-010), human MMP9 (R&D Systems: 911-MP-010), and mouse MMP9 (R&D Systems: 909-MM-010), were used to evaluate linker cleavage of 3′ PIP-MMPa-CpG and 3′ PIP-MMPb-CpG conjugates. MMP enzymes were activated with 1 mM APMA in assay buffer (50 mM Tris, 10 mM CaCl₂, 150 mM NaCl, pH 7.5) at 37° C. for the appropriate length of time specified by the manufacturer. Activated MMP enzymes (final concentration=3 nM) were then incubated with 5 μM conjugates (i.e., 3′ PIP-MMPa-CpG and 3′ PIP-MMPb-CpG) at 37° C. for 2 h in assay buffer (50 μL per sample). Samples were then frozen and stored at −20° C. On a separate day, denaturing PAGE gels (15%) were prepared using the SequaGel UreaGel 19:1 Denaturing Gel System. To prepare samples for gel analyses, samples were thawed, diluted in 1× denaturing loading buffer, and incubated at 95° C. for 2 min. After heating, samples were immediately put on ice and then loaded onto the gel. Gels were run at constant 15 W for 30 minutes using 1×TBE as the electrophoresis buffer. Gels were then stained using SYBR™ Gold Nucleic Acid Gel Stain.

Enzymatically-Cleavable Linkers Remain Functional after 3′ Class C CpG Conjugation:

As described in the previous example, 3′ PIP-MMPa-CpG and 3′ PIP-MMPb-CpG contain MMP2/MMP9-cleavable peptide linkers. After localizing to the tumor through the tumor-targeting domain (i.e., PIP), these MMP-cleavable conjugates should allow CpG to be released in the tumor microenvironment, which could facilitate uptake by surrounding immune cells. To verify these MMP-cleavable linkers retained their intended function after conjugation, the 3′ PIP-MMPa-CpG and 3′ PIP-MMPb-CpG were evaluated in MMP2/9 enzyme cleavage assays.

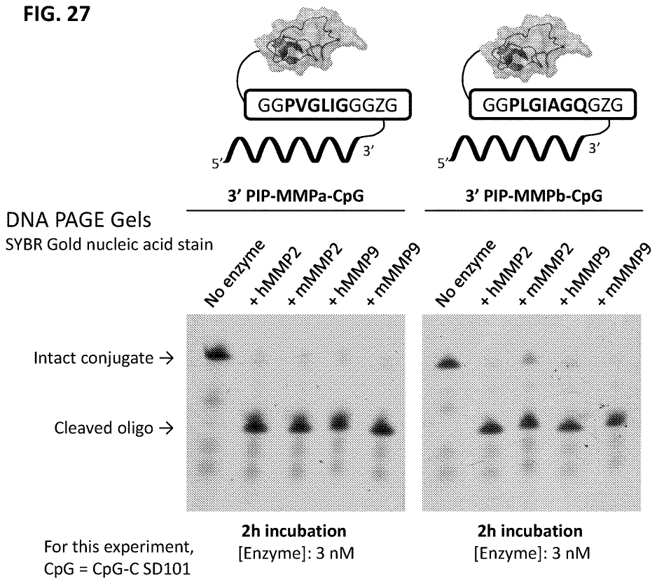

FIG. 27 shows that 3′ PIP-MMPa-CpG and 3′ PIP-MMPb-CpG conjugates are efficiently cleaved by human and mouse MMP2 and MMP9.

### Example 8—for Tumor-Targeting CpG Conjugates, the CpG Conjugation Site does not Affect Binding Affinity of the Tumor-Targeting Agent

### Competition Binding Assays:

To compare the relative binding affinities of PIP and various PIP-CpG conjugates, cell-based competition binding assays were performed using human PANC1 pancreatic cancer cells. Alexa Fluor 647-labeled PIP-Fc fusion (PIP-Fc-AF647) was used as a fluorescent binder to compare the binding affinity of unlabeled competitors. To avoid possible ligand depletion mediated through DNA-binding interactions, PANC1 cells (50,000 cells/sample) were pretreated with 500 nM of unmodified CpG for 10 min at 4° C. in 100 μL of integrin-binding buffer (IBB: 25 mM Tris pH 7.4, 150 mM NaCl, 2 mM CaCl₂, 1 mM MgCl₂, 1 mM MnCl₂, and 0.5% BSA). Without washing, pretreated PANC1 cells (50,000 cells/sample) were then incubated in the dark with 0.5 nM PIP-Fc-AF647 and varying concentrations of unlabeled ligands for 2 h at 4° C. in a final volume of 200 μL/sample (resulting concentration of unmodified CpG=250 nM). A positive control containing only the fluorescent binder (0.5 nM PIP-Fc-AF647; no unlabeled competitor) and a negative control with only IBB (no fluorescent binder or competitor) were included. Cells were kept on ice or at 4° C. throughout the remainder of the assay. Following incubation, cells were washed three times with cold PBSA (PBS+0.5% BSA) and analyzed on a BD Accuri C6 Plus flow cytometer. The median fluorescence intensity (MFI) from each sample was determined, and the dataset was normalized such that the MFI from the positive control was equal to 100% Bound, and the MFI from the negative control was equal to 0% Bound. Error bars represent the SD of 3 replicates.

### New 3′ PIP-CpG Conjugates Retain Similar Binding Affinity as the Unconjugated PIP:

To verify the various 3′ PIP-CpG conjugates also retained the tumor-binding capacity of PIP after conjugation, the cleavable conjugates were evaluated in a competition binding assay.

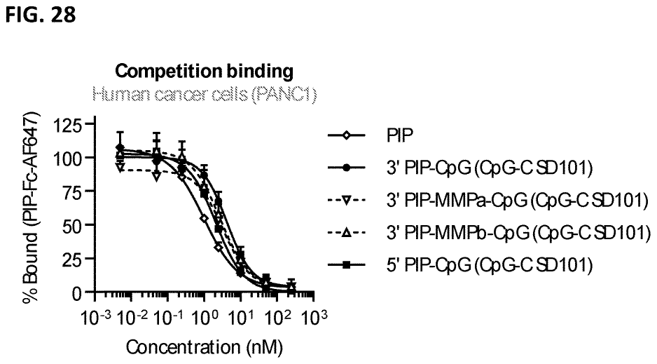

FIG. 28 shows the results of a competition binding assay performed with human PANC1 pancreatic cancer cells; these data demonstrate that 3′ PIP-CpG, 3′ PIP-MMPa-CpG, 3′ PIP-MMPb-CpG, and 5′ PIP-CpG retain similar binding affinity as the unconjugated PIP peptide.

These results demonstrate that PIP binding capacity is broadly conserved for different PIP-CpG conjugates.

### REFERENCES

1. Miller, C. L. et al. Systemic delivery of a targeted synthetic immunostimulant transforms the immune landscape for effective tumor regression. Cell Chemical Biology 29, 451-462.e8 (2022).
2. Putta, M. R. et al. Peptide Conjugation at the 5′-End of Oligodeoxynucleotides Abrogates Toll-Like Receptor 9-Mediated Immune Stimulatory Activity. Bioconjugate Chem. 21, 39-45 (2010).
3. Yu, D., Zhao, Q., Kandimalla, E. R. & Agrawal, S. Accessible 5′-end of CpG-containing Phosphorothioate Oligodeoxynucleotides is essential for immunostimulatory activity. Bioorganic & Medicinal Chemistry Letters 10, 2585-2588 (2000).
4. Kramer, K., Young, S. L. & Walker, G. F. Comparative Study of 5′- and 3′-Linked CpG-Antigen Conjugates for the Induction of Cellular Immune Responses. ACS Omega 2, 227-235 (2017).
5. Ignacio, B. J., Albin, T. J., Esser-Kahn, A. P. & Verdoes, M. Toll-like Receptor Agonist Conjugation: A Chemical Perspective. Bioconjugate Chemistry 29, 587-603 (2018).
6. Sagiv-Barfi, I., Levy, R., Miller, C., Cochran, J. R., Bertozzi, C. R. Knottin-immunostimulant conjugates and related compositions and methods. WO2021067261A1. 2021.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A conjugate comprising:
a targeting moiety or antigen conjugated via a linker to the 3′ end region of a CpG oligonucleotide,
wherein the CpG oligonucleotide is one which, in its unconjugated state, activates NF-κB and interferon regulatory factor (IRF) pathways.

2. The conjugate of claim 1, wherein the CpG oligonucleotide comprises palindromic sequences.

3. The conjugate of claim 2, wherein the palindromic sequence comprises a first sequence and a second sequence, wherein the second sequence is a reverse complement of the first sequence and wherein the first sequence and second sequence are each from 4 to 50, 4 to 25, 4 to 20, 5, 6, 7, 8, 9, 10, 11, 12, 13, or 14 nucleotides long.

4. The conjugate of any one of claims 1 to 3, wherein the CpG oligonucleotide comprises from 10 to 100, from 10 to 50, or from 20 to 40 nucleotides.

5. The conjugate of any one of claims 1 to 4, wherein the CpG oligonucleotide comprises CpG dinucleotides with 1, 2, 3, 4, 5, 6, or 7 intervening nucleotides between each CpG dinucleotide, independently.

6. The conjugate of any one of claims 1 to 5, wherein the targeting moiety or antigen is conjugated to the 5th nucleotide, the 4th nucleotide, the 3rd nucleotide, or the 2nd nucleotide from the 3′ end, or the last nucleotide on the 3′ end of the CpG oligonucleotide.

7. The conjugate of claim 6, wherein the targeting moiety or antigen is conjugated to the last nucleotide on the 3′ end of the CpG oligonucleotide.

8. The conjugate of any one of claims 1 to 7, wherein the CpG oligonucleotide comprises one or more internucleotide linkages resistant to nuclease degradation.

9. The conjugate of claim 8, wherein the one or more internucleotide linkages resistant to nuclease degradation comprise a phosphorothioate linkage, a boranophosphate linkage, a phosphorodiamidate linkage, a phosphoamidate linkage, a thiophosphoramidate linkage, or any combination thereof.

10. The conjugate of claim 8, wherein the one or more internucleotide linkages resistant to nuclease degradation comprise one or more phosphorothioate linkages.

11. The conjugate of any one of claims 1 to 10, wherein the CpG oligonucleotide is a class-C CpG oligonucleotide.

12. The conjugate of any one of claims 1 to 11, wherein the CpG oligonucleotide comprises the following nucleotide sequence: 5′-TCGAACGTTCGAACGTTCGAACGTTCGAAT-3′. (SEQ ID NO: 1).

13. The conjugate of any one of claims 1 to 11, wherein the CpG oligonucleotide comprises the following nucleotide sequence: 5′-TCGTCGTCGTTCGAACGACGTTGAT-3′. (SEQ ID NO: 2).

14. The conjugate of any one of claims 1 to 13, comprising a targeting moiety conjugated to the 3′ end region of a CpG oligonucleotide.

15. The conjugate of claim 14, wherein the targeting moiety is chosen from: a polypeptide, a ligand, an aptamer, a nanoparticle, and a small molecule.

16. The conjugate of claim 15, wherein the targeting moiety is a polypeptide.

17. The conjugate of claim 16, wherein the targeting moiety comprises an antibody.

18. The conjugate of claim 17, wherein the antibody is an IgG, single chain Fv (scFv), Fab, (Fab)2, (scFv′)2, or a single variable domain located on a heavy chain (VHH).

19. The conjugate of any one of claims 14 to 18, wherein the targeting moiety specifically binds a cell surface molecule.

20. The conjugate of any one of claims 14 to 19, wherein the targeting moiety specifically binds to a tumor antigen on the surface of the cancer cell.

21. The conjugate of claim 20, wherein the tumor antigen is αvβ1 integrin, αvβ3 integrin, αvβ5 integrin, αvβ6 integrin, α5β1 integrin, 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAPi), tissue factor (TF), T cell immunoglobulin and mucin protein-1 (TIM-1), Tn antigen, trophoblast cell-surface antigen (TROP-2), Wilms' tumor 1 (WT1), or VEGF-A, or any combination thereof.

22. The conjugate of any one of claims 14 to 21, wherein the targeting moiety specifically binds αvβ1 integrin, αvβ3 integrin, αvβ5 integrin, αvβ6 integrin, α5β1 integrin, or any combination thereof.

23. The conjugate of any one of claims 14 to 22, wherein the targeting moiety is a knottin peptide.

24. The conjugate of claim 23, wherein the knottin peptide comprises an EETI-II peptide, an AgRP peptide, a ω-conotoxin peptide, a Kalata B1 peptide, an MCoTI-II peptide, an agatoxin peptide, or a chlorotoxin peptide.

25. The conjugate of claim 23 wherein the knottin peptide comprises an EETI-II peptide.

26. The conjugate of any one of claims 23 to 25, wherein the knottin peptide is an EETI-based integrin-binding peptide comprising GCX₁X₂X₃X₄X₅X₆X₇X₈X₉X₁₀X₁₁X₁₂X₁₃X₁₄X₁₅X₁₆X₁₇X₁₈X₁₉X₂₀CX₂₁QDSDCX₂₂AGCVCGPNGX₂₃CG (SEQ ID NO: 19) wherein X₁-X₃ are any amino acid; X₄-X₂₀, if present, are any amino acid; and wherein X₂₁-X₂₃ are any amino acid, further wherein each amino acid is independently selected from standard or unnatural amino acids.

27. The conjugate of any one of claims 23 to 26, wherein the knottin peptide comprises an integrin binding loop comprising PRPRGDNPPLT (SEQ ID NO: 22) or PQGRGDWAPTS (SEQ ID NO: 23).

28. The conjugate of any one of claims 14 to 27, wherein the targeting moiety comprises a ligand, a nanoparticle, or a small molecule.

29. The conjugate of any one of claims 1 to 13, comprising an antigen conjugated to the 3′ end region of a CpG oligonucleotide.

30. The conjugate of claim 29, wherein the antigen is a microbial antigen.

31. The conjugate of claim 29, wherein the microbial antigen is a bacterial antigen.

32. The conjugate of claim 29, wherein the antigen is a viral antigen.

33. The conjugate of claim 29, wherein the antigen is a tumor antigen.

34. The conjugate of claim 29, wherein the antigen is an allergen.

35. The conjugate of any one of claims 1 to 34, wherein the linker is a non-cleavable linker.

36. The conjugate of any one of claims 1 to 34, wherein the linker is a cleavable linker.

37. The conjugate of claim 36, wherein the linker is an enzymatically cleavable linker.

38. A composition comprising the conjugate of any one of claims 1 to 37.

39. The composition of claim 38, wherein the composition is formulated for administration to a subject in need thereof.

40. The composition of claim 39, comprising the conjugate of any one of claims 20 to 22.

41. A method comprising administering to a subject in need thereof an effective amount of the composition of claim 39.

42. A method of treating cancer in a subject in need thereof, the method comprising administering to the subject the composition of claim 40 in an amount effective to treat the cancer.

43. The method of claim 42, wherein the cancer is chosen from a solid tumor, optionally a blastoma, carcinoma, lymphoma, or sarcoma.

44. The method according to claim 41 to 43, wherein the subject is receiving an immune checkpoint inhibitor therapy.

45. The method according to claim 44, wherein the immune checkpoint inhibitor therapy comprises administration to the subject of a therapeutically effective amount of an inhibitor of B7-H3, CTLA-4, LAG-3, PD-1, PD-L1, TIGIT, TIM-3, VISTA, or any combination thereof.

46. The method according to claim 45, wherein the immune checkpoint inhibitor therapy comprises administration to the subject of a therapeutically effective amount of a PD-1 inhibitor.

47. The method according to claim 46, wherein the PD-1 inhibitor is an anti-PD-1 antibody.

48. The method according to claim 47, wherein the anti-PD-1 antibody is camrelizumab, cemiplimab, dostarlimab, nivolumab, pembrolizumab, pidilizumab, pimivalimab, retifanlimab, sintilimab, spartalizumab, tislelizumab, or toripalimab.

49. The method according to any one of claims 44 to 48, wherein the immune checkpoint inhibitor therapy comprises administration to the subject of a therapeutically effective amount of a PD-L1 inhibitor.

50. The method according to claim 49, wherein the PD-L1 inhibitor is an anti-PD-L1 antibody.

51. The method according to claim 50, wherein the anti-PD-L1 antibody is atezolizumab, avelumab, or durvalumab.

52. The method according to any one of claims 44 to 51, comprising administering to the subject the immune checkpoint inhibitor therapy.

53. An immunogenic composition comprising the conjugate of any one of claims 29 to 34, and a pharmaceutically acceptable carrier.

54. A method of inducing an immune response in a subject, the method comprising administering the immunogenic composition of claim 53 to the subject an amount effective to induce an immune response in the subject.


SEQUENCE LISTING (46 sequences)

SEQ ID NO: 1 - DNA, 30 bp, synthetic construct
  source 1..30: mol_type = other DNA
tcgaacgttcgaacgttcgaacgttcgaat

SEQ ID NO: 2 - DNA, 25 bp, synthetic construct
  source 1..25: mol_type = other DNA
tcgtcgtcgttcgaacgacgttgat

SEQ ID NO: 3 - DNA, 22 bp, synthetic construct
  source 1..22: mol_type = other DNA
tcgtcgttttcggcgcgcgccg

SEQ ID NO: 4 - DNA, 23 bp, synthetic construct
  source 1..23: mol_type = other DNA
tcgtaacgttcgaacgttcgaac

SEQ ID NO: 5 - DNA, 21 bp, synthetic construct
  source 1..21: mol_type = other DNA
tcgtcgaacgttcgagatgat

SEQ ID NO: 6 - DNA, 25 bp, synthetic construct
  source 1..25: mol_type = other DNA
tcgaacgttcgaacgttcgaacgtt

SEQ ID NO: 7 - DNA, 29 bp, synthetic construct
  source 1..29: mol_type = other DNA
tcgcgaacgttcgccgcgttcgaacgcgg

SEQ ID NO: 8 - DNA, 24 bp, synthetic construct
  source 1..24: mol_type = other DNA
tcgtcgttttgtcgttttgtcgtt

SEQ ID NO: 9 - DNA, 22 bp, synthetic construct
  source 1..22: mol_type = other DNA
tgactgtgaacgttcgagatga

SEQ ID NO: 10 - DNA, 20 bp, synthetic construct
  source 1..20: mol_type = other DNA
tccatgacgttcctgacgtt

SEQ ID NO: 11 - DNA, 20 bp, synthetic construct
  source 1..20: mol_type = other DNA
tccatgacgttcctgatgct

SEQ ID NO: 12 - DNA, 20 bp, synthetic construct
  source 1..20: mol_type = other DNA
ggtgcatcgatgcagggggg

SEQ ID NO: 13 - DNA, 20 bp, synthetic construct
  source 1..20: mol_type = other DNA
gggggacgatcgtcgggggg

SEQ ID NO: 14 - DNA, 21 bp, synthetic construct
  source 1..21: mol_type = other DNA
ggggacgacgtcgtggggggg

SEQ ID NO: 15 - DNA, 20 bp, synthetic construct
  source 1..20: mol_type = other DNA
ggggtcaacgttgagggggg

SEQ ID NO: 16 - DNA, 30 bp, synthetic construct
  source 1..30: mol_type = other DNA
gggggggggggacgatcgtcgggggggggg

SEQ ID NO: 17 - protein, 28 aa, synthetic construct
GCPRILMRCKQDSDCLAGCVCGPNGFCG

SEQ ID NO: 18 - protein, 33 aa, synthetic construct
GCPRPRGDNPPLTCZQDSDCLAGCVCGPNGYCG

SEQ ID NO: 19 - protein, 42 aa, synthetic construct
  VARIANT 3..5: note = wherein X is any amino acid, including standard or unnatural amino acids
  VARIANT 6..22: note = wherein X is any amino acid, including standard or unnatural amino acids, or may be deleted
  VARIANT 24: note = wherein X is any amino acid, including standard or unnatural amino acids
  VARIANT 30: note = wherein X is any amino acid, including standard or unnatural amino acids
  VARIANT 40: note = wherein X is any amino acid, including standard or unnatural amino acids
GCXXXXXXXXXXXXXXXXXXXXCXQDSDCXAGCVCGPNGXCG

SEQ ID NO: 20 - protein, 11 aa, synthetic construct
TGRGDSPASSK

SEQ ID NO: 21 - protein, 11 aa, synthetic construct
VTGRGDSPASS

SEQ ID NO: 22 - protein, 11 aa, synthetic construct
PRPRGDNPPLT

SEQ ID NO: 23 - protein, 11 aa, synthetic construct
PQGRGDWAPTS

SEQ ID NO: 24 - protein, 11 aa, synthetic construct
TIGRGDWAPSE

SEQ ID NO: 25 - protein, 33 aa, synthetic construct
GCPRPRGDNPPLTCZQDSDCLAGCVCGPNGFCG

SEQ ID NO: 26 - protein, 33 aa, synthetic construct
GCPRPRGDNPPLTCSQDSDCLAGCVCGPNGFCG

SEQ ID NO: 27 - protein, 33 aa, synthetic construct
GCPRPRGDNPPLTCSQDSDCLAGCVCGPNGYCG

SEQ ID NO: 28 - protein, 33 aa, synthetic construct
GCPRPRGDNPPLTCKQDSDCLAGCVCGPNGFCG

SEQ ID NO: 29 - protein, 33 aa, synthetic construct
GCPRPRGDNPPLTCKQDSDCLAGCVCGPNGYCG

SEQ ID NO: 30 - protein, 33 aa, synthetic construct
GCPQGRGDWAPTSCSQDSDCRAGCVCGPNGFCG

SEQ ID NO: 31 - protein, 33 aa, synthetic construct
GCPQGRGDWAPTSCSQDSDCRAGCVCGPNGYCG

SEQ ID NO: 32 - protein, 33 aa, synthetic construct
GCPQGRGDWAPTSCZQDSDCRAGCVCGPNGFCG

SEQ ID NO: 33 - protein, 33 aa, synthetic construct
GCPQGRGDWAPTSCZQDSDCRAGCVCGPNGYCG

SEQ ID NO: 34 - protein, 33 aa, synthetic construct
GCPQGRGDWAPTSCSQDSDCLAGCVCGPNGFCG

SEQ ID NO: 35 - protein, 33 aa, synthetic construct
GCPQGRGDWAPTSCSQDSDCLAGCVCGPNGYCG

SEQ ID NO: 36 - protein, 33 aa, synthetic construct
GCPQGRGDWAPTSCZQDSDCLAGCVCGPNGFCG

SEQ ID NO: 37 - protein, 33 aa, synthetic construct
GCPQGRGDWAPTSCZQDSDCLAGCVCGPNGYCG

SEQ ID NO: 38 - protein, 34 aa, synthetic construct
CVRLHESCLGQQVPCCDPAATCYCRFFNAFCYCR

SEQ ID NO: 39 - protein, 29 aa, synthetic construct
CGETCVGGTCNTPGCTCSWPVCTRNGLPV

SEQ ID NO: 40 - protein, 34 aa, synthetic construct
SGSDGGVCPKILKKCRRDSDCPGACICRGNGYCG

SEQ ID NO: 41 - protein, 36 aa, synthetic construct
MCMPCFTTDHQMARKCDDCCGGKGRGKCYGPQCLCR

SEQ ID NO: 42 - protein, 33 aa, synthetic construct
GCPQGRGDWAPTSCKQDSDCRAGCVCGPNGFCG

SEQ ID NO: 43 - protein, 33 aa, synthetic construct
GCPQGRGDWAPTSCKQDSDCRAGCVCGPNGYCG

SEQ ID NO: 44 - protein, 33 aa, synthetic construct
  VARIANT 15: note = wherein X is an unnatural amino acid bearing an azide group
GCPRPRGDNPPLTCXQDSDCLAGCVCGPNGYCG

SEQ ID NO: 45 - protein, 12 aa, synthetic construct
GGPVGLIGGGZG

SEQ ID NO: 46 - protein, 12 aa, synthetic construct
GGPLGIAGQGZG